(19) United States
(12) Patent Application Publication
(10) Pub. No.: US 2025/0282770 A1
(43) Pub. Date: Sep. 11, 2025

(54) BIOLUMINESCENT PROBES TO TRACK STEM CELLS IN VIVO

(71) Applicant: THE BOARD OF TRUSTEES OF THE UNIVERSITY OF ILLINOIS, Urbana, IL (US)

(72) Inventors: Jefferson Kar Fai CHAN, Savoy, IL (US); Chelsea SWARTCHICK, Urbana, IL (US)

(21) Appl. No.: 19/069,369

(22) Filed: Mar. 4, 2025

Related U.S. Application Data
(60) Provisional application No. 63/561,450, filed on Mar. 5, 2024.

(51) Int. Cl. C07D 417/04; A61P 35/00; C07D 491/107

(52) U.S. Cl. CPC C07D 417/04; A61P 35/00; C07D 491/107

(57) ABSTRACT

In this work, we have developed a luciferin derivative, AlDeLuc, the first logic-gated bioluminescence probe for cancer stem cells (CSCs) that is sequentially activated by acidic environments and ALDH1A1 activity. The reliance on these two biomarkers is critical to ensure that off-target detection of non-CSCs within the body is minimized. Beyond demonstrating efficacy in multiple cancer cell lines and a murine model of breast cancer, we employed AlDeLuc to investigate how the CSC population is altered by the inflammatory status of a tumor through a high-fat diet. The implication of this work provides a molecular link between obesity, inflammation, and tumor progression.

### RELATED APPLICATIONS

This application claims priority under 35 U.S.C. § 119(e) to U.S. Provisional Patent Application No. 63/561,450, filed Mar. 5, 2024, which is incorporated herein by reference.

### BACKGROUND OF THE INVENTION

Cancer stem cells (CSCs) are a small subset of cells within a malignant tumor characterized by their unique ability to self-renew and differentiate. In the context of cancer relapse, residual CSCs that have escaped eradication can repopulate a tumor site with cancer cells exhibiting highly aggressive and treatment-resistant phenotypes. For instance, breast CSCs are believed to be the major culprit of poor clinical outcomes and low therapeutic response amongst breast cancer patients. CSCs are commonly identified based on the overexpression of aldehyde dehydrogenase 1A1 (ALDH1A1), which belongs to a superfamily of 19 ALDH enzymes, each capable of catalyzing the oxidation of xenobiotic and endogenous aldehydes to their corresponding carboxylates. In addition to its cytoprotective role, ALDH1A1 controls CSC differentiation through the retinoic acid signaling pathway. However, the ability to identify CSCs in an in vivo setting during cancer progression and treatment is challenging with the use of existing ALDH1A1 technology. For instance, ALDH1A1 is an intracellular target, precluding the use of antibodies required for live cell staining or immunohistochemical methods. Likewise, the use of small-molecule fluorescent indicators such as ALDEFLUOR in live animals is not recommended as this results in off-target staining due to the prevalence of ALDH in the body, as well as cross-reactivity with non-ALDH1A1 isoforms. While the recent development of fluorogenic (turn-on) probes that exhibit ALDH1A1 isoform selectivity has overcome this specific barrier, background interference due to autofluorescence associated with this modality limits CSC detection to cell cultures, excised tissue samples, or superficially within tumors. On the other hand, bioluminescence imaging is characterized by an inherent low background. Moreover, when augmented with biomarker-responsive probes, this modality is primed for detecting rare events and low-abundance cell populations such as CSCs.

Traditional bioluminescence probes are constructed based on capping of the phenolic alcohol of luciferin with simple enzyme substrates such as phosphate or galactose for alkaline phosphatase (ALP) or beta-galactosidase, respectively. The presence of these moieties blocks the interaction of a probe with luciferase, and thus, no bioluminescence is generated prior to their removal. However, these classic examples can only detect simple enzyme targets and not small molecules (e.g., nitric oxide, hydrogen peroxide) or redox-active metal ions (e.g., copper) that are important to cancer biology. In contrast, modern bioluminescence probes are designed utilizing the principles of activity-based sensing (ABS), which, beyond abundance, also considers how the chemical reactivity of a biomarker can impact the bioluminescence readout in a given local environment at a particular instance in time. For example, the activity of ALDH1A1 can be influenced by intracellular pH fluxes, availability of the NAD+ cofactor, concentration of substrate and products, and so forth. Indeed, ABS probes for an impressive list of enzymes, reactive oxygen species, reactive nitrogen species, biological thiols, and metal ions have been developed. However, to detect CSCs via ALDH1A1 activity, it is imperative to also consider potential interference by non-CSCs such as normal stem cells, which are also characterized by the overexpression of this enzyme. This poses a significant concern because a probe can, in theory, be activated by normal stem cells prior to its localization to a tumor site. Therefore, the resulting bioluminescence may not accurately report on local ALDH1A1 activity of CSCs.

Accordingly, there is a need for a bioluminescent probe that is suitable for in-vivo detection of cancer stem cells.

### SUMMARY

This disclosure provides a bioluminescent probe that is activated via interaction with two sequential CSC biomarkers in vivo. The probe must first respond to acid to become a substrate for ALDH1A1. Subsequently, a luciferase substrate is liberated that is oxidized to yield a bioluminescent signal.

Accordingly, this disclosure provides a compound of formula I:

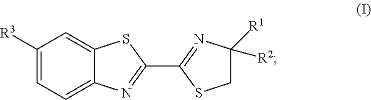

or a salt thereof,
wherein
   R¹ is —CH(OR^{a})₂, —CHO, or —CO₂R^{b};
   R² is H; or
      R¹ and R² taken together with the carbon atom to which they are attached is:

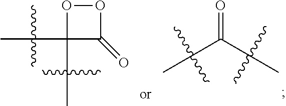

R³ is —N(R^{c})² or —SR;
R^{a} is each independently —(C₁-C₆)alkyl or H;
R^{b} is H or adenosine monophosphate (AMP); and
R^{c} is each independently —(C₁-C₆)alkyl;
wherein each —(C₁-C₆)alkyl can independently be unbranched —(C₃-C₆)alkyl or branched —(C₃-C₆)alkyl.

This disclosure also provides a method for detecting cancer stem cells (CSCs), comprising:
a) contacting CSCs comprising an aldehyde dehydrogenase (ALDH), an acid, and a compound of formula I; and
b) detecting a bioluminescent signal emitted from the CSCs;
wherein, a compound of formula I comprising the acetal moiety R¹ is —CH(OR^{a})₂ is hydrolyzed by the acid to an aldehyde, and the aldehyde is oxidized by the ALDH to liberate a carboxylic acid substrate for luciferase; wherein the bioluminescent signal emitted from the CSCs is thereby detected.

The invention provides novel compounds of Formulas I, II, III, IIIA, IV, and IVA, intermediates for the synthesis of compounds of Formulas I, II, III, IIIA, IV, and IVA, as well as methods of preparing compounds of Formulas I, II, III, IIIA, IV, and IVA. The invention also provides compounds of Formulas I, II, III, IIIA, IV, and IVA that are useful as intermediates for the synthesis of other useful compounds.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1A-F. a) Bioluminescent images of compounds 9, 10, and 11 (100 μM) after 60 min incubation with 1 M HCl, treatment with ALDH1A1 (0.4 μM), and luciferase. b) Quantified data from a. c) In vitro assay demonstrating all components: AlDeLuc, acid-hydrolysis, ALDH1A1, and luciferase, must be present to generate bioluminescence. d) Bioluminescent images demonstrating signal intensity is dependent on the concentration of AlDeLuc present. e) Quantified data from d. f) Bioluminescent signal after AlDeLuc (5 μM) is incubated with various amounts of ALDH1A1. Luciferase concentration was held constant at 0.02 mg/mL for all experiments. Statistical analysis was performed using two-tailed Student's t-test with Welch's correction (α=0.05), ***ρ<0.001, ****ρ<0.0001.

FIG. 2A-B. a) Normalized bioluminescence of AlDeLuc (2 μM) upon incubation with each ALDH isoform after 60 min at room temperature. (n=5) b) Response of AlDeLuc (50 μM) after incubation with various reactive oxygen and nitrogen species, biological thiols, and amines at concentrations of 100 μM. Cys and GSH were tested at 1 mM. ALDH1A1 activation is provided for reference. Luciferase (0.005 mg/mL) was added to initiate bioluminescence production. (n=3).

FIG. 3A-B. a) Representative images of MSC or A549 cell lysates after treatment with AlDeLuc for 1 h, then treatment with luciferase. b) Representative bioluminescent images of A549 cell lysates after cotreatment with NCT-501 (ALDH1A1-specific inhibitor) (40 μM) and AlDeLuc (20 μM) for 1 h followed by addition of luciferase. Luciferase concentration held constant at 0.05 mg/mL and n=4 for all experiments. Statistical analysis was performed using two-tailed Student's t test with Welch's correction (α=0.05), **ρ<0.01.

FIG. 4A-D. a) Representative bioluminescent images of 4T1 cell lysates incubated with AlDeLuc (100 μM) for 60 minutes then administered luciferase. Blank=protease buffer. b) Representative bioluminescence images of 4T1 lysates after cells were pretreated with diethylaminobenzaldehyde (DEAB) (500 μM 0.5% DMSO final concentration) or vehicle (0.5% DMSO), followed by a 1 hour incubation with a mixture of AlDeLuc (100 μM) and DEAB (500 μM) or AlDeLuc alone (100 μM) (final DMSO concentration 1%), then administered luciferase. c) Representative images of 4T1 cells pretreated with L-NMMA (1 mM) or vehicle, followed by AlDeLuc (50 μM) and luciferase. d) Representative luminescent images of 4T1 cell lysates pretreated with INF-γ (26.8 ng/mL) or vehicle for 12 hours, then administered AlDeLuc (50 μM) and luciferase. Luciferase concentration held constant at 0.05 mg/mL and n=4 for all experiments. Statistical analysis was performed using two-tailed Student's t-test with Welch's correction (α=0.05), **ρ<0.01.

FIG. 5A-H. a) Timeline of the AlDeLuc systemic toxicity investigation. b) Hemoxatoxylin and eosin stains of the major organs from mice administered either AlDeLuc or vehicle to evaluate the biocompatibility of our probe. Scale bar represents 50 m. c) Representative bioluminescent images of BALB/c mice bearing 4T1-Luc tumors acquired 40 min after systemic administration of AlDeLuc (left) and two days following a mixture of AlDeLuc and DEAB (right). d) Quantified data from c. e) Timeline of high-fat diet (HFD) induced-inflammatory murine model generated to explore the influence of NO on ALDH1A1 activity using AlDeLuc. f) Representative bioluminescent images of BALB/c mice bearing orthotopic mammary 4T1-Luc tumors after a 20-week HFD priming period that were either administered L-NMMA (50 mg/kg) (left) or water (right) via oral gavage daily for three weeks, and were retro-orbitally administered AlDeLuc (0.55 mg/kg). Images were collected 40 min after injection. g) Quantified data from f. h) Immunohistochemical stains of 4T1-Luc tumors with iNOS or ALDH1A1 antibodies from mice treated with L-NMMA (+L-NMMA) or vehicle (−L-NMMA). Statistical analysis was performed using two-tailed Student's t-test with Welch's correction (α=0.05), **ρ<0.01. Scale bar represents 50 m.

FIG. 6. Absorption spectrum of AlDeLuc at 100 μM in DMSO (green), and the luminescent spectra of AlDeLuc after sequential acid activation, ALDH1A1 oxidation, and luciferase turnover.

FIG. 7. AlDeLuc (2 μM) after a one-hour incubation in 1 M HCl, 1 M NaCl, or 1 M NaOH to demonstrate the acid-activation required to yield a bioluminescent signal. Statistical analysis was performed using two-tailed Student's t-test with Welch's correction (α=0.05), ****ρ<0.0001, NS=not significant.

FIG. 8. Bioluminescent data showing AlDeLuc does not hydrolyze in serum-containing media (red) or PBS (green), compared to acid-activated AlDeLuc in PBS (blue).

FIG. 9. The stability of AlDeLuc (10 μM) after incubation with 1 unit of β-Galactosidase (β-gal). Statistical analysis was performed using two-tailed Student's t-test with Welch's correction (α=0.05), **ρ<0.01.

FIG. 10. The stability of AlDeLuc (2 μM) after a 30-minute incubation with increasing concentrations of rat-liver microsomes (RLM). Statistical analysis was performed using two-tailed Student's t-test with Welch's correction (α=0.05), NS=not significant.

FIG. 11. Absorbance spectrum at 340 nm demonstrating NADH production when ALDH1A1 (0.4 μM) is incubated with AlDeLuc (2 μM) in the presence of NAD⁺ (2 mM) in triethanolamine buffer (50 mM, pH 7.4), (n=3).

FIG. 12A-B. a) Representative bioluminescent images of A549 cell lysates (˜3.6×10₆ cells) after incubation with AlDeLuc (100 μM) for 60 min and subsequent treatment with luciferase. Blank=protease buffer. Luciferase concentration held constant at 0.05 mg/mL and n=4. Statistical analysis was performed using two-tailed Student's t-test with Welch's correction (α=0.05), ***ρ<0.001. b) Bioluminescence signal intensity of A549-Luc cells pre-treated with either NCT-501 (40 μM) or vehicle for 30 min, followed by incubation with AlDeLuc (20 μM) at 37° C. Luciferase concentration held constant at 0.05 mg/mL and n=3. Statistical analysis was performed using two-tailed Student's t-test with Welch's correction (α=0.05), ****r<0.001.

FIG. 13. Cell viability for AlDeLuc in 4T1-Luc cells after 2 hours as measured by the MTT assay. Values are reported as mean±standard deviation (n=3).

FIG. 14A-D. a) 4T1-Luc cells administered BL₆₆₀NO (NO-responsive bioluminescent probe; 39 μM) after incubation with DEA-NONOate (50 μM) for 30 minutes. b) Quantified data from a. c) 4T1-Luc cells administered BL₆₆₀NO (20 μM) after incubation with L-NMMA (1 mM) for 30 minutes. d) Quantified data from c. Statistical analysis was performed using two-tailed Student's t-test with Welch's correction (α=0.05), *ρ<0.05.

FIG. 15. 4T1 cells after 2-day treatment with vehicle or DETA NONOate (50 μM) supplemented media, followed by incubation with AlDeLuc (2 μM). Statistical analysis was performed using two-tailed Student's t-test with Welch's correction (α=0.05), **ρ<0.01.

FIG. 16A-B. a) BALB/c mice bearing 4T1-Luc tumors 40 minutes after retroorbital administration of PBS (left) and AlDeLuc (0.55 mg/kg, 1:19 v/v DMSO:PBS) (right). b) Quantified data from a (n=10, two tumors per five mice). Statistical analysis was performed using two-tailed Student's t-test with Welch's correction (α=0.05), ****ρ<0.0001.

FIG. 17. Representative image of BALB/c mouse bearing 4T1-Luc tumors 40 minutes after retroorbital (RO) administration of AlDeLuc (0.55 mg/kg, 1:19 v/v DMSO:PBS) (right) the same subject 24 hours after RO administration demonstrating complete clearance.

### DETAILED DESCRIPTION

Although cancer stem cells (CSCs) represent only a small subpopulation of a malignant tumor, they have a disproportionate impact on chemotherapeutic resistance and cancer relapse. CSCs are commonly identified by the overexpression of aldehyde dehydrogenase 1A1 (ALDH1A1), which, in addition to their protective roles against reactive aldehydes, controls differentiation through the retinoic acid signaling pathway. However, methods to identify CSCs through this biomarker in vivo are scarce. Beyond their low prevalence, properties of the tumor microenvironment (TME) are hypothesized to influence the number of CSCs, as well as their sternness profile. In this work, we have developed AlDeLuc, the first logic-gated bioluminescence probe for CSCs that is sequentially activated by acidic environments and ALDH1A1 activity. The reliance on these two biomarkers is critical to ensure that off-target detection of non-CSCs within the body is minimized. Beyond demonstrating efficacy in multiple cancer cell lines and a murine model of breast cancer, we employed AlDeLuc to investigate how the CSC population is altered by the inflammatory status of a tumor through a high-fat diet. The implication of this work provides a molecular link between obesity, inflammation, and tumor progression.

Additional information and data that can be used with aspects of the invention described herein can be found in the publication by the inventors Swartchick et al, JACS Au 2025, 5, 1, 320-331 and its Supporting Information, which are incorporated herein by reference in their entirety.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number1” to “number2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number10”, it implies a continuous range that includes whole numbers and fractional numbers less than number10, as discussed above. Similarly, if the variable disclosed is a number greater than “number10”, it implies a continuous range that includes whole numbers and fractional numbers greater than number10. These ranges can be modified by the term “about”, whose meaning has been described above.

The recitation of a), b), c), . . . or i), ii), iii), or the like in a list of components or steps do not confer any particular order unless explicitly stated.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

As used herein, “subject” or “patient” means an individual having symptoms of, or at risk for, a disease or other malignancy. A patient may be human or non-human and may include, for example, animal strains or species used as “model systems” for research purposes, such a mouse model as described herein. Likewise, the patient may include either adults orjuveniles (e.g., children).

Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods provided herein, the mammal is a human.

As used herein, the terms “providing”, “administering,” “introducing,” are used interchangeably herein and refer to the placement of a compound of the disclosure into a subject by a method or route that results in at least partial localization of the compound to a desired site. The compound can be administered by any appropriate route that results in delivery to a desired location in the subject.

The compound and compositions described herein may be administered with additional compositions to prolong stability and activity of the compositions, or in combination with other therapeutic drugs.

The term “substantially” as used herein, is a broad term and is used in its ordinary sense, including, without limitation, being largely but not necessarily wholly that which is specified. For example, the term could refer to a numerical value that may not be 100% the full numerical value.

The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation or limitations which is not specifically disclosed herein.

This disclosure provides methods of making the compounds and compositions of the invention. The compounds and compositions can be prepared by any of the applicable techniques described herein, optionally in combination with standard techniques of organic synthesis. Many techniques such as etherification and esterification are well known in the art. However, many of these techniques are elaborated in Compendium of Organic Synthetic Methods (John Wiley & Sons, New York), Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, Jr., 1980; Vol. 5, Leroy G. Wade, Jr., 1984; and Vol. 6; as well as standard organic reference texts such as March's Advanced Organic Chemistry: Reactions, Mechanisms, and Structure, 5th Ed., by M. B. Smith and J. March (John Wiley & Sons, New York, 2001); Comprehensive Organic Synthesis. Selectivity, Strategy & Efficiency in Modern Organic Chemistry. In 9 Volumes, Barry M. Trost, Editor-in-Chief (Pergamon Press, New York, 1993 printing); Advanced Organic Chemistry, Part B: Reactions and Synthesis, Second Edition, Cary and Sundberg (1983); for heterocyclic synthesis see Hermanson, Greg T., Bioconjugate Techniques, Third Edition, Academic Press, 2013.

The formulas and compounds described herein can be modified using protecting groups. Suitable amino and carboxy protecting groups are known to those skilled in the art (see for example, Protecting Groups in Organic Synthesis, Second Edition, Greene, T. W., and Wutz, P. G. M., John Wiley & Sons, New York, and references cited therein; Philip J. Kocienski; Protecting Groups (Georg Thieme Verlag Stuttgart, New York, 1994), and references cited therein); and Comprehensive Organic Transformations, Larock, R. C., Second Edition, John Wiley & Sons, New York (1999), and referenced cited therein.

The term “halo” or “halide” refers to fluoro, chloro, bromo, or iodo. Similarly, the term “halogen” refers to fluorine, chlorine, bromine, and iodine.

The term “alkyl” refers to a branched or unbranched hydrocarbon having, for example, from 1-20 carbon atoms, and often 1-12, 1-10, 1-8, 1-6, or 1-4 carbon atoms; or for example, a range between 1-20 carbon atoms, such as 2-6, 3-6, 2-8, or 3-8 carbon atoms. As used herein, the term “alkyl” also encompasses a “cycloalkyl”, defined below. Examples include, but are not limited to, methyl, ethyl, 1-propyl, 2-propyl (iso-propyl), 1-butyl, 2-methyl-1-propyl (isobutyl), 2-butyl (sec-butyl), 2-methyl-2-propyl (t-butyl), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, hexyl, octyl, decyl, dodecyl, and the like. The alkyl can be unsubstituted or substituted, for example, with a substituent described below or otherwise described herein. The alkyl can also be optionally partially or fully unsaturated. As such, the recitation of an alkyl group can include an alkenyl group or an alkynyl group. The alkyl can be a monovalent hydrocarbon radical, as described and exemplified above, or it can be a divalent hydrocarbon radical (i.e., an alkylene).

An alkylene is an alkyl group having two free valences at a carbon atom or two different carbon atoms of a carbon chain. Similarly, alkenylene and alkynylene are respectively an alkene and an alkyne having two free valences at two different carbon atoms, or an alkenylene can have the two free valences on the same carbon.

The term “cycloalkyl” refers to cyclic alkyl groups of, for example, from 3 to 10 carbon atoms having a single cyclic ring or multiple condensed rings. Cycloalkyl groups include, by way of example, single ring structures such as cyclopropyl, cyclobutyl, cyclopentyl, cyclooctyl, and the like, or multiple ring structures such as adamantyl, and the like. The cycloalkyl can be unsubstituted or substituted. The cycloalkyl group can be monovalent or divalent and can be optionally substituted as described for alkyl groups. The cycloalkyl group can optionally include one or more cites of unsaturation, for example, the cycloalkyl group can include one or more carbon-carbon double bonds, such as, for example, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, and the like.

The term “heteroatom” refers to any atom in the periodic table that is not carbon or hydrogen. Typically, a heteroatom is O, S, N, P. The heteroatom may also be a halogen, metal or metalloid.

The term “heterocycloalkyl” or “heterocyclyl” refers to a saturated or partially saturated monocyclic, bicyclic, or polycyclic ring containing at least one heteroatom selected from nitrogen, sulfur, oxygen, preferably from 1 to 3 heteroatoms in at least one ring. Each ring is preferably from 3- to 10-membered, more preferably 4 to 7 membered. Examples of suitable heterocycloalkyl substituents include pyrrolidyl, tetrahydrofuryl, tetrahydrothiofuranyl, piperidyl, piperazyl, tetrahydropyranyl, morpholino, 1,3-diazapane, 1,4-diazapane, 1,4-oxazepane, and 1,4-oxathiapane. The group may be a terminal group or a bridging group.

The term “aryl” refers to an aromatic hydrocarbon group derived from the removal of at least one hydrogen atom from a single carbon atom of a parent aromatic ring system. The radical attachment site can be at a saturated or unsaturated carbon atom of the parent ring system. The aryl group can have from 6 to 30 carbon atoms, for example, about 6-10 carbon atoms. The aryl group can have a single ring (e.g., phenyl) or multiple condensed (fused) rings, wherein at least one ring is aromatic (e.g., naphthyl, dihydrophenanthrenyl, fluorenyl, or anthryl). Typical aryl groups include, but are not limited to, radicals derived from benzene, naphthalene, anthracene, biphenyl, and the like. The aryl can be unsubstituted or optionally substituted with a substituent described below. For example, a phenyl moiety or group may be substituted with one or more substituents R^{X} where R^{X} is at the ortho-, meta-, orpara-position, and X is an integer variable of 1 to 5.

The term “heteroaryl” refers to a monocyclic, bicyclic, or tricyclic ring system containing one, two, or three aromatic rings and containing at least one nitrogen, oxygen, or sulfur atom in an aromatic ring. The heteroaryl can be unsubstituted or substituted, for example, with one or more, and in particular one to three, substituents, as described in the definition of “substituted”. Typical heteroaryl groups contain 2-20 carbon atoms in the ring skeleton in addition to the one or more heteroatoms, wherein the ring skeleton comprises a 5-membered ring, a 6-membered ring, two 5-membered rings, two 6-membered rings, or a 5-membered ring fused to a 6-membered ring. Examples of heteroaryl groups include, but are not limited to, 2H-pyrrolyl, 3H-indolyl, 4H-quinolizinyl, acridinyl, benzo[b]thienyl, benzothiazolyl, β-carbolinyl, carbazolyl, chromenyl, cinnolinyl, dibenzo[b,d]furanyl, furazanyl, furyl, imidazolyl, imidizolyl, indazolyl, indolisinyl, indolyl, isobenzofuranyl, isoindolyl, isoquinolyl, isothiazolyl, isoxazolyl, naphthyridinyl, oxazolyl, perimidinyl, phenanthridinyl, phenanthrolinyl, phenarsazinyl, phenazinyl, phenothiazinyl, phenoxathiinyl, phenoxazinyl, phthalazinyl, pteridinyl, purinyl, pyranyl, pyrazinyl, pyrazolyl, pyridazinyl, pyridyl, pyrimidinyl, pyrrolyl, quinazolinyl, quinolyl, quinoxalinyl, thiadiazolyl, thianthrenyl, thiazolyl, thienyl, triazolyl, tetrazolyl, and xanthenyl. In one embodiment the term “heteroaryl” denotes a monocyclic aromatic ring containing five or six ring atoms containing carbon and 1, 2, 3, or 4 heteroatoms independently selected from non-peroxide oxygen, sulfur, and N(Z) wherein Z is absent or is H, O, alkyl, aryl, or (C₁-C₆)alkylaryl. In some embodiments, heteroaryl denotes an ortho-fused bicyclic heterocycle of about eight to ten ring atoms derived therefrom, particularly a benz-derivative or one derived by fusing a propylene, trimethylene, or tetramethylene diradical thereto.

As used herein, the term “substituted” or “substituent” is intended to indicate that one or more (for example, in various embodiments, 1-10; in other embodiments, 1-6; in some embodiments 1, 2, 3, 4, or 5; in certain embodiments, 1, 2, or 3; and in other embodiments, 1 or 2) hydrogens on the group indicated in the expression using “substituted” (or “substituent”) is replaced with a selection from the indicated group(s), or with a suitable group known to those of skill in the art, provided that the indicated atom's normal valency is not exceeded, and that the substitution results in a stable compound. Suitable indicated groups include, e.g., alkyl, alkenyl, alkynyl, alkoxy, haloalkyl, hydroxyalkyl, aryl, heteroaryl, heterocyclyl, cycloalkyl, alkanoyl, alkoxycarbonyl, amino, alkylamino, dialkylamino, carboxyalkyl, alkylthio, alkylsulfinyl, and alkylsulfonyl. Substituents of the indicated groups can be those recited in a specific list of substituents described herein, or as one of skill in the art would recognize, can be one or more substituents selected from alkyl, alkenyl, alkynyl, alkoxy, halo, haloalkyl, hydroxy, hydroxyalkyl, aryl, heteroaryl, heterocycle, cycloalkyl, alkanoyl, alkoxycarbonyl, amino, alkylamino, dialkylamino, trifluoromethylthio, difluoromethyl, acylamino, nitro, trifluoromethyl, trifluoromethoxy, carboxy, carboxyalkyl, keto, thioxo, alkylthio, alkylsulfinyl, alkylsulfonyl, and cyano. Suitable substituents of indicated groups can be bonded to a substituted carbon atom include F, Cl, Br, I, OR′, OC(O)N(R′)₂, CN, CF₃, OCF₃, R′, O, S, C(O), S(O), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂NHC(O)R′, N(R′)N(R′)C(O)R′, N(R′)N(R′)C(O)OR′, N(R′)N(R′)CON(R′)₂, N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety (e.g., (C₁-C₆)alkyl), and wherein the carbon-based moiety can itself be further substituted. When a substituent is monovalent, such as, for example, F or Cl, it is bonded to the atom it is substituting by a single bond. When a substituent is divalent, such as O, it is bonded to the atom it is substituting by a double bond; for example, a carbon atom substituted with O forms a carbonyl group, C═O.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof, such as racemic mixtures, which form part of the present invention. Many organic compounds exist in optically active forms, i.e., they have the ability to rotate the plane of plane-polarized light. In describing an optically active compound, the prefixes D and L, or R and S. are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate (defined below), which may occur where there has been no stereoselection or stereospecificity in a chemical reaction or process.

The terms “racemic mixture” and “racemate” refer to an equimolar mixture of two enantiomeric species, devoid of optical activity.

### Statements of the Technology

1. A compound of formula I:

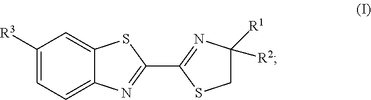

or a salt thereof,
wherein
   R¹ is —CH(OR^{a})₂, —CHO, —C(═O)R^{b}, or —CO₂R^{b};
   R² is H; or
      R¹ and R² taken together with the carbon atom to which they are attached is:

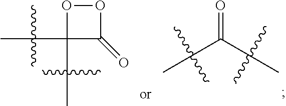

R³ is —N(R^{c})², —OR^{c}, or —SR^{c};
R^{a} is each independently —(C₁-C₆)alkyl or H;
R^{b} is H, —(C₁-C₆)alkyl, or adenosine monophosphate (AMP); and
R^{c} is each independently —(C₁-C₆)alkyl;
wherein each —(C₁-C₆)alkyl can independently be unbranched —(C₃-C₆)alkyl or branched —(C₃-C₆)alkyl.
2. The compound of statement 1, wherein R¹ is —CH(OR^{a})₂.
3. The compound of statement 1 or 2, wherein each R^{a} is —CH₃, —CH₂CH₃, or —CH(CH₃)₂.
4. The compound of any one of statements 1-3, wherein R³ is —N(R^{c})².
5. The compound of any one of statements 1-4, wherein each R^{c} is —CH₂CH₃.
6. The compound of any one of statements 1-5, wherein the carbon atom to which R¹ and R² are attached has an (S)-configuration.
7. The compound of any one of statements 1-5, wherein the carbon atom to which R¹ and R² are attached has an (R)-configuration.
8. The compound of any one of statements 1-7, represented by formula II:

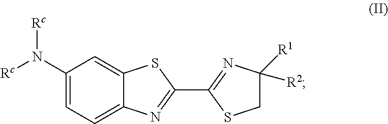

or a salt thereof.
9. The compound of any one of statements 1-7, represented by formula III or IIIA:

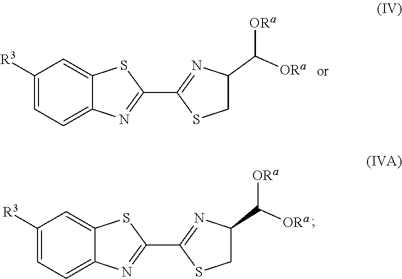

or a salt thereof.
10. The compound of any one of statements 1-7, represented by formula IV or IVA:

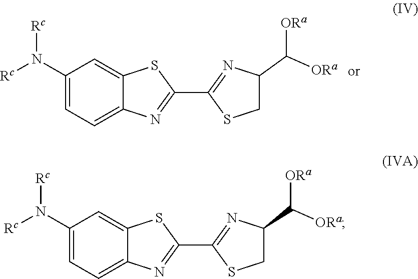

or a salt thereof.
11. The compound of statement 1, wherein the compound is AlDeLuc:

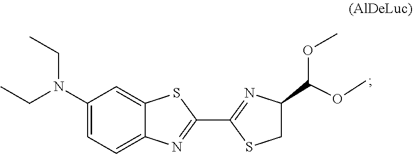 [SMILES: CCN(CC)c1ccc2nc(C3=N[C@@H](C(OC)OC)CS3)sc2c1]

or a salt thereof.
12. The compound of statement 1, wherein the compound is:

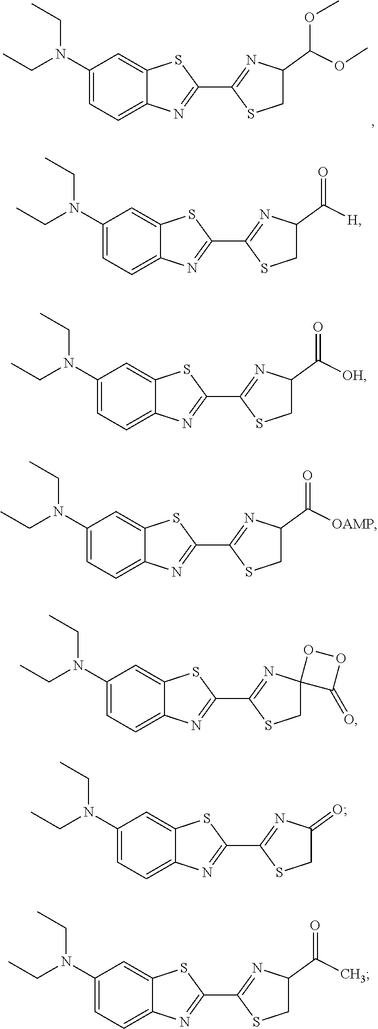

or a salt thereof.
13. A composition comprising a compound of any one of statements 1-12 and pharmaceutically acceptable diluent or carrier.
14. A method for detecting cancer stem cells (CSCs), comprising:
   a) contacting CSCs comprising an aldehyde dehydrogenase (ALDH), an acid, and a compound of formula I:

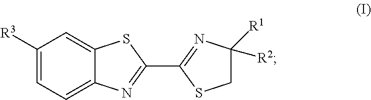

or a salt thereof,
wherein
   R¹ is —CH(OR^{a})₂, —CHO, —C(═O)R^{b}, or —CO₂R^{b}.
   R² is H; or
      R¹ and R² taken together with the carbon atom to which they are attached is:

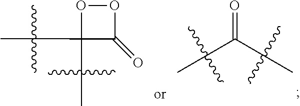

R³ is —N(R^{c})², —OR^{c}, or —SR^{c};
R^{a} is each independently —(C₁-C₆)alkyl or H;
R^{b} is H, —(C₁-C₆)alkyl, or adenosine monophosphate (AMP); and
R^{c} is each independently —(C₁-C₆)alkyl;
wherein each —(C₁-C₆)alkyl can independently be unbranched —(C₃-C₆)alkyl or branched —(C₃-C₆)alkyl; and
   b) detecting a bioluminescent signal emitted from the CSCs;
   wherein, a compound of formula I comprising the acetal moiety R¹ is —CH(OR^{a})₂ is hydrolyzed by the acid to an aldehyde, and the aldehyde is oxidized by the ALDH to liberate a carboxylic acid substrate for luciferase;
   wherein the bioluminescent signal emitted from the CSCs is thereby detected.
15. The method of statement 14, wherein the compound is (S)-2-(4-(dimethoxymethyl)-4,5-dihydrothiazol-2-yl)-N,N-diethylbenzo[d]thiazol-6-amine (AlDeLuc).
16. The method of statement 14 or 15, wherein the aldehyde dehydrogenase is the ALDH₁A₁ isoform.
17. The method of any one of statements 14-16, wherein the luciferase catalyzes a conversion of the carboxylic acid substrate to 2-(6-(diethylamino)benzo[d]thiazol-2-yl)thiazol-4(5H)-one that yields a bioluminescent signal.
18. The method of any one of statements 14-17, wherein a tumor comprises the cancer stem cells,
19. The method of statement 18, wherein the tumor comprises the acid in a tumor microenvironment (TME) or an endosomal compartment within the cancer stem cells.
20. The method of any one of statements 14-19, wherein the aldehyde is 2-(6-(diethylamino)benzo[d]thiazol-2-yl)-4,5-dihydrothiazole-4-carbaldehyde and/or the carboxylic acid substrate is 2-(6-(diethylamino)benzo[d]thiazol-2-yl)-4,5-dihydrothiazole-4-carboxylic acid.
21. The compound, composition or method of any one of statements 1, 13, or 14, wherein the compound is:

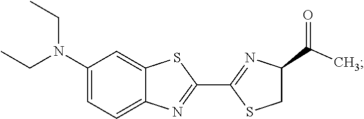 [SMILES: CCN(CC)c1ccc2nc(C3=N[C@@H](C(C)=O)CS3)sc2c1]

or a salt thereof.
21. The compound, composition or method of statements 21, wherein the compound (control-AlDeLuc) is a negative-control of the aldehyde form of AlDeLuc after its acetal trigger has been removed hydrolytically via acid treatment;
   wherein as opposed to the aldehyde form, the ketone is not a substrate of ALDH1A1, and the ketone is not capable of generating bioluminescence in the presence of luciferase.

### Activity-Based Logic-Gate Probe Reveals Crosstalk Between the Inflammatory Tumor Microenvironment and the ALDH1A1 Cancer Stem Cell Biomarker

In this work, we rationally developed logic-gated bioluminescence probe, AlDeLuc, that can only be activated via interaction with two sequential CSC biomarkers. While CSCs are well-documented to reside in acidic tumor microenvironments (TME), normal stem cells do not, which provides us with an ‘and-gate’ to differentiate between the two. Specifically, AlDeLuc must first respond to acid prior to ALDH1A1, and only then will the luciferase substrate be liberated (Scheme 1). Beyond demonstrating excellent selectivity over other common ALDH isoforms, reactive biological species, and a robust signal turn-on when both biomarkers are present, we also showcased its utility in multiple cancer cell lines, as well as in vivo using a murine model of breast cancer. Enabled by the excellent performance of our probe, we performed additional experiments in cytokine-activated cultured cancer cells and chronically obese animals given a high-fat diet to explore a potential molecular link between inflammation and the stemness profile of CSCs as determined by ALDH1A1 activity.

### Scheme 1. Schematic Showing the Logic-Gated Activation of AlDeLuc to Yield a Bioluminescence Signal in Response to Sequential Exposure to Acid and ALDH1A1 Activity

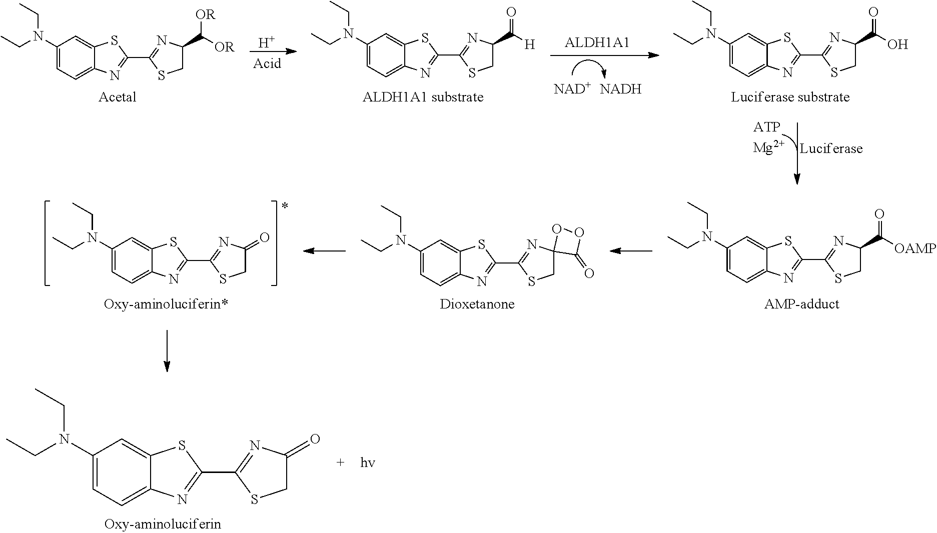

### Results and Discussion

Design and Synthesis of Logic-Gate Probes. To achieve the requisite properties to detect ALDH1A1 activity within CSCs, which only accounts for ˜0.01-2% of cells within a solid tumor, three critical design criteria were prioritized. First, besides ALDH1A1, the body expresses 18 other ALDH isoforms that are each capable of oxidizing various aldehyde substrates to their corresponding carboxylic acid products. Based on our previous work in developing various isoform-selective ABS probes (ACS Central Science 2018, 4, 1045-1055), we discovered that in the case of ALDH1A1, reactivity is favored when the aldehyde moiety is attached to cyclic structures such as a phenyl ring due to their geometric fit within the enzyme binding pocket. Therefore, we hypothesized that substituting the carboxylic acid group of firefly luciferin at the 4,5-dihydrothiazole core with an aldehyde would impart comparable selectivity for the ALDH1A1 isoform. Moreover, because the carboxylic acid is essential for luciferase recognition, binding and turnover, the aldehyde would therefore represent an inert trigger. Second, anticipating that ALDH1A1 activity may occur at sites other than at the tumor (e.g., hepatic stem cells), we further converted the aldehyde into an acid-responsive acetal group. This design exploits the acidic TME, as well as abundant acidic endosomes in cancer cells to mediate site-specific probe activation. Lastly, we strategically replaced the pH-sensitive phenol with a pH-insensitive N,N-diethyl aniline to ensure that the bioluminescence signal will remain constant. With these criteria in mind, we devised a modular synthetic route to access the requisite building blocks for the proposed logic-gated probe for ALDH1A1.

First, 2-chlorobenzothiazole was subjected to standard electrophilic aromatic substitution conditions to yield the mono-nitrated intermediate 1 in 74% yield. A Beauchamp reduction was then employed to convert the aromatic nitro group to the corresponding aniline 2 in 67%, which allowed for subsequent installation of ethyl groups via reductive amination to afford the N,N-diethylamino intermediate 3 in 55% yield. Treatment of 3 with potassium cyanide afforded the cyano-benzothiazole 4 via SNAr chemistry in 82% yield. In parallel, D-cysteine hydrochloride was reacted with dimethoxypropane in acetone to furnish the 2,2-dimethylthiazolidine intermediate 5 in 83% yield. The secondary amine was Boc-protected to yield 6, and its carboxylic acid group was transformed to the Weinreb-amide intermediate 7 in 35% yield over two-steps. Lithium aluminum hydride was then employed to access the aldehyde 8 in 77% yield. With this in hand, we attempted to mask the aldehyde as an acetal, which was followed by deprotection of the boc-group and 2,2-dimethylthiazolidine core. This sequence was initially unsuccessful as the major product formed was an imidic acid by-product. After extensive screening, we found its formation could be suppressed by rigorously degassing the alcohol solvent used for the acetalization step, as well as performing deprotection of the 2,2-dimethylthiazolidine under an inert nitrogen atmosphere. After two days, the solvent could then be exchanged for degassed THF containing 4 to give the desired acetal product(s). By varying the alcohol solvent (i.e., methanol, ethanol, and isopropanol), we were able to access 9, 10, or 11 (Scheme 2).

### Scheme 2. Synthetic Route to Yield Panel of Logic-Gated Bioluminescent Probes for ALDH1A1 Activity. R=Me, Et, or iPr

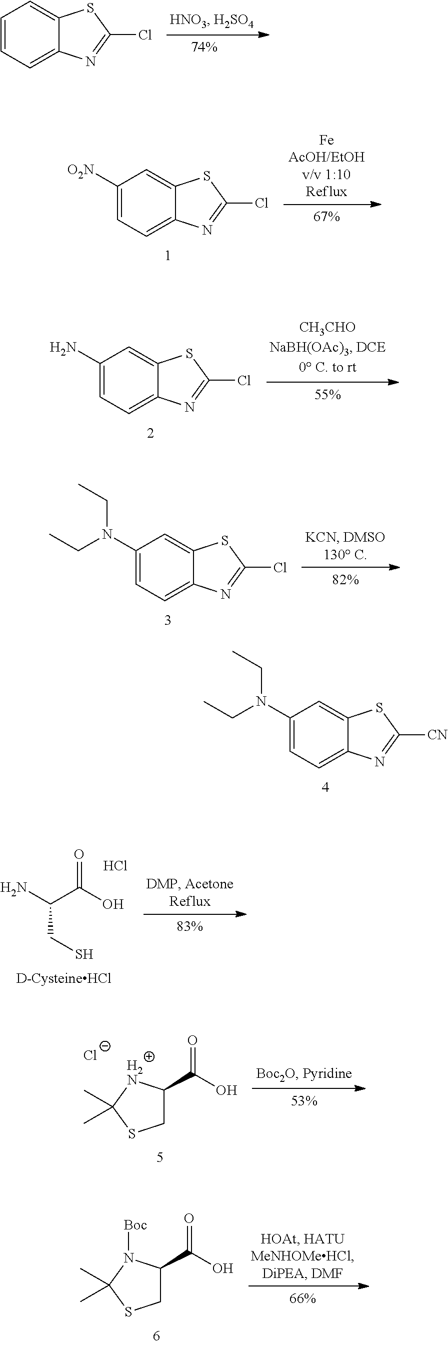

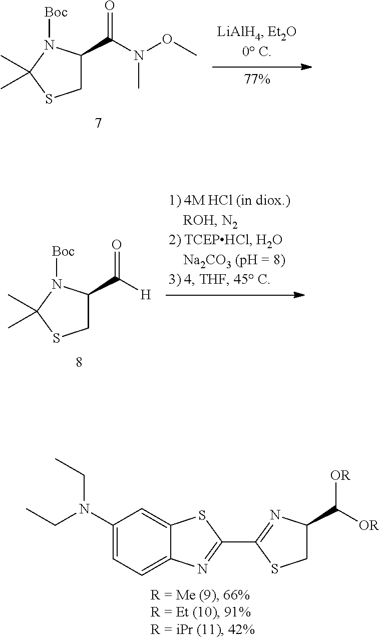

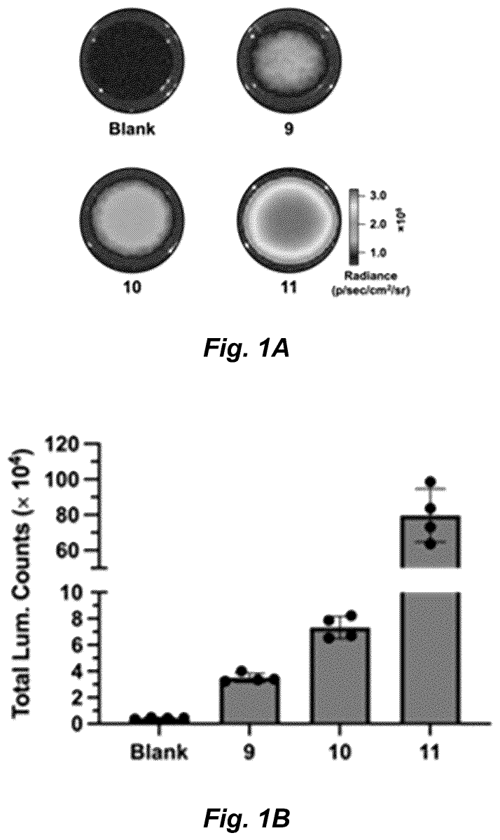

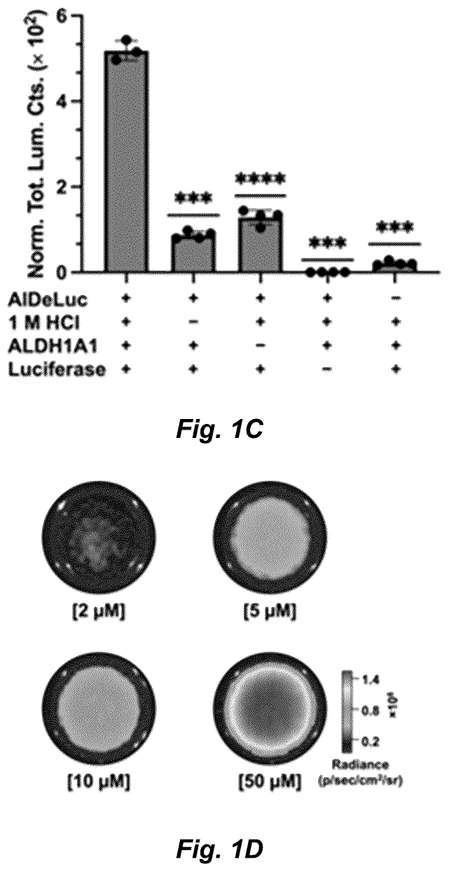

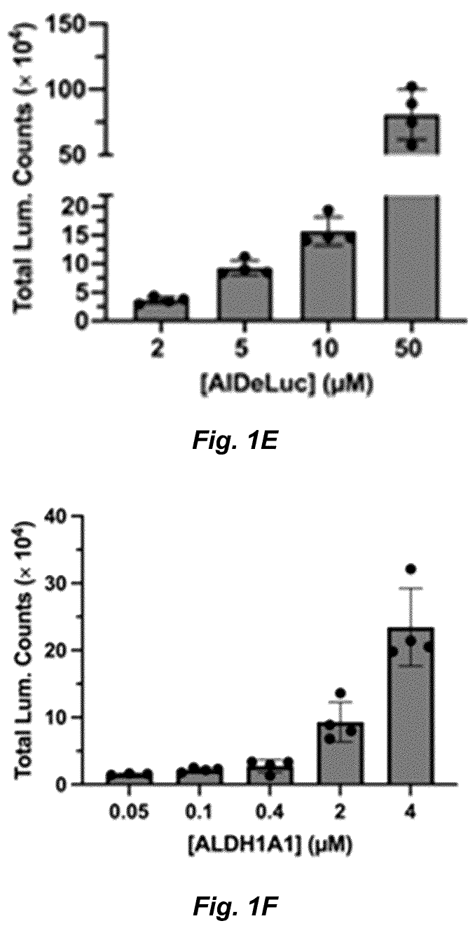

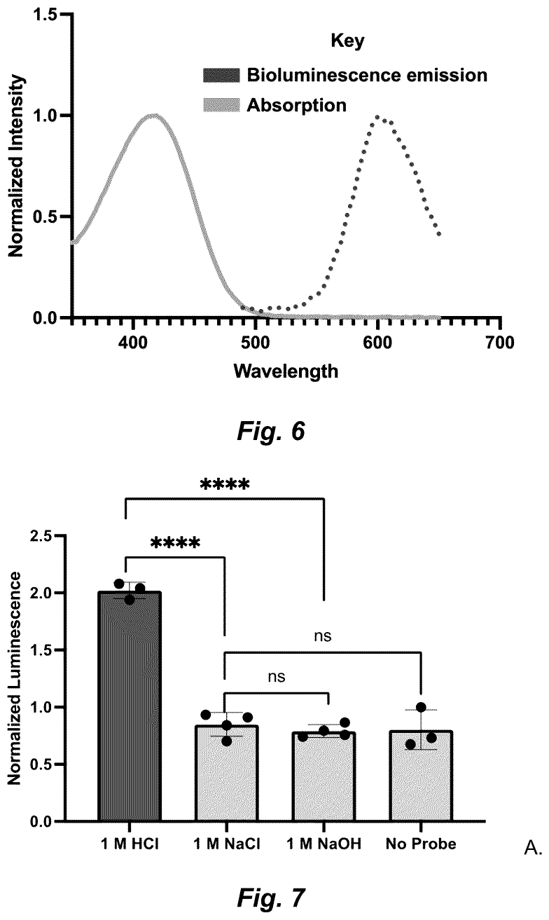

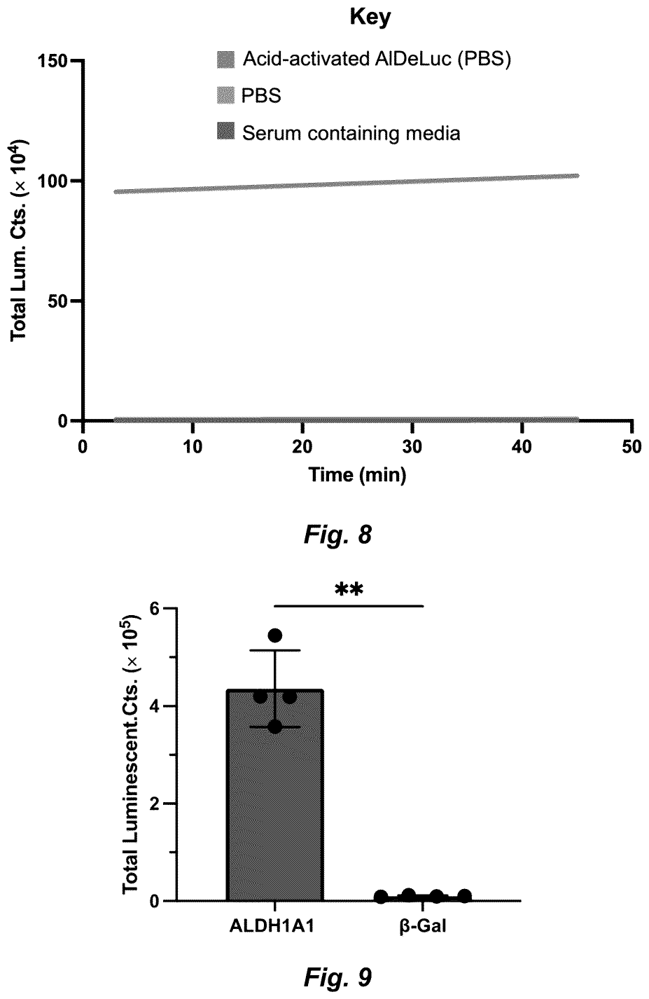

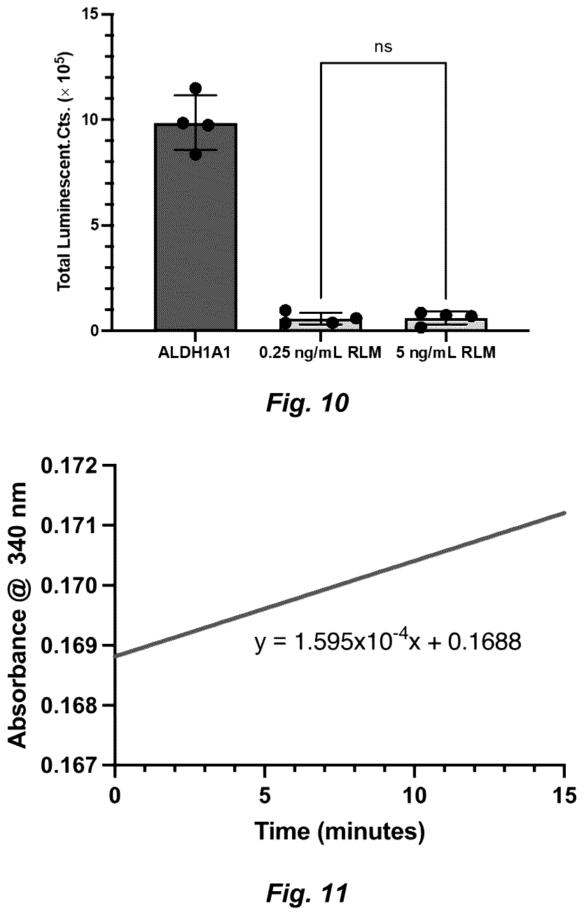

In Vitro Evaluation of Acetal Trigger Stability. After obtaining 9, 10 and 11, our goal was to first identify the most acid-resistant acetal to ensure stability while in circulation. Although our results indicate that each probe can be readily deprotected upon incubation in 1 M HCl for one hour, 9 was the most acid-resistant as the bioluminescence emission was lowest after neutralization and treatment with ALHD1A1 and luciferase (λₑₘ=605 nm at pH 7.4 and temp.=37° C.) (FIGS. 1a, 1b and 6). Additionally, we observed no bioluminescence when 9 was treated with 1 M NaCl or 1 M NaOH, demonstrating no acetal unmasking at neutral or alkaline pH values, respectively (FIG. 7). Likewise, incubation in PBS or serum containing media yielded identical results (FIG. 8). We also examined potential off-target activation by glucosidases (e.g., beta-galactosidase (β-Gal)) owing to their ability to hydrolyze acetals to hemiacetals, which in the case of 9 will further decompose to yield an aldehyde substrate. Even in the presence of 1 unit of β-Gal, the acetal remained completely stable (FIG. 9). Lastly, we treated 9 with rat liver microsomes at various concentrations to account for possible CYP450 activity that have been reported to cleave acetals. Across a range of concentrations from 0.25 to as high as 5.0 ng/mL, negligible bioluminescence was observed (FIG. 10). Collectively, these results indicate that the dimethyl acetal satisfies our logic-gate design criteria and thus, 9 will herein be referred to as AlDeLuc.

Mechanism of AlDeLuc Logic-Gate Activation. Next, to examine the mode of AlDeLuc activation, we performed assays where exposure to acidic conditions and ALDH1A1 were investigated separately (FIG. 1c). Compared to when both conditions were applied, the signal of AlDeLuc upon treatment with luciferase was significantly attenuated, which is consistent with the envisioned logic-gate mechanism. Likewise, when either AlDeLuc or luciferase was excluded, the bioluminescent signal was also negligible. Next, we varied the concentration of AlDeLuc from 2 to 50 μM and observed a concentration-dependent increase in signal intensity (FIGS. 1d and 1e). Similarly, when the concentration of AlDeLuc was held constant and the amount of ALDH1A1 was varied (from 0 to 4 μM), we noted that the wells with the highest ALDH1A1 concentration showed the most intense signal (Figure if).

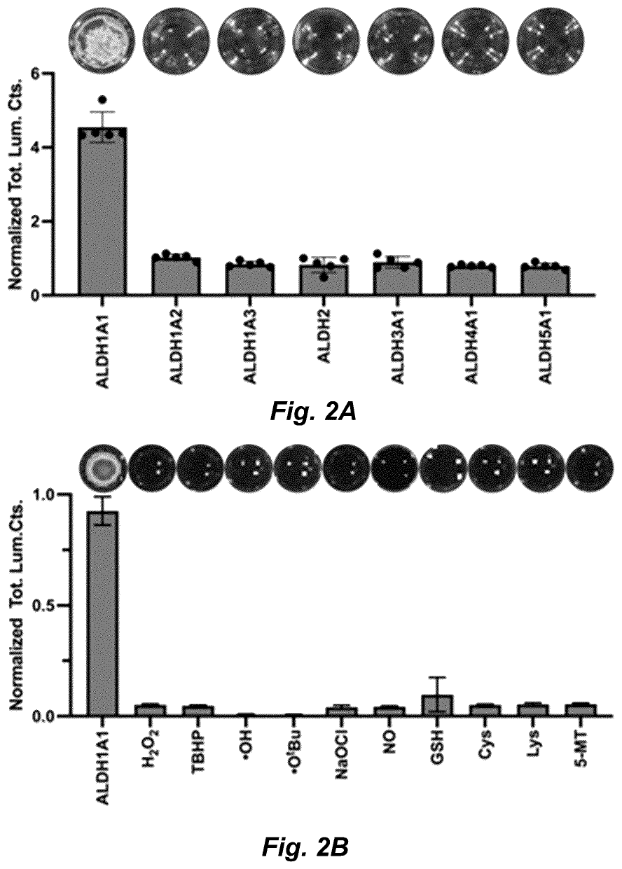

Although these results collectively show that AlDeLuc is a competent substrate for ALDH1A1 after acetal removal, it is critical to further examine potential cross-reactivity with other common ALDH isoforms. After expressing and purifying ALDH1A2, ALDH1A3, ALDH2, ALDH3A1, ALDH4A1, and ALDH5A1, we normalized their enzymatic activity using their preferred aldehyde substrates. AlDeLuc was then pre-activated with HCl, neutralized, and incubated with each of the isoforms under identical conditions. To our delight, only treatment with ALDH1A1 and luciferase generated significant bioluminescence (FIG. 2a). We corroborated these results by monitoring the absorbance change at 340 nm, which is indicative of NADH production during aldehyde oxidation (FIG. 11). Beyond testing common ALDH isoforms, we assessed the potential oxidation of the aldehyde to the corresponding carboxylic acid due to high levels of reactive oxygen species in cancer cells. No interference was found upon treatment of the aldehyde form of AlDeLuc with peroxides (e.g., H₂O₂, tert-Butyl hydroperoxide), radicals (e.g., ·OH, ·OtBu), or hypochlorite (FIG. 2b). Lastly, we examined whether the presence of glutathione and amino acids such as lysine and cysteine would attenuate ALDH1A1-catalyzed oxidation due to their ability to form Schiff bases or thiazolidines, respectively. Additionally, we tested 5-methoxytryptamine, which is known to react with aldehydes via Pictet-Spengler chemistry. The presence of these analytes did not attenuate the bioluminescence, compared to control reactions where they were excluded.

Evaluation of AlDeLuc in Bone Marrow Mesenchymal Stem Cells and A549 Human Lung Cancer Cells. Our next objective was to evaluate the performance of AlDeLuc in live cells to assess uptake, activation, and possible interference from components of the complex cellular milieu. For this purpose, we obtained mesenchymal stem cells (MSCs) (CD34+, ALDH1A1+) from a commercial vendor (ATCC). We followed recommended culturing procedures to obtain ˜2.5×10⁵ cells that were undifferentiated and viable. MSCs were selected as the noncancerous counterpart because they overexpress ALDH1A1 and in comparison to cancer cells, are known to exhibit a more neutral pH within their intracellular compartments.

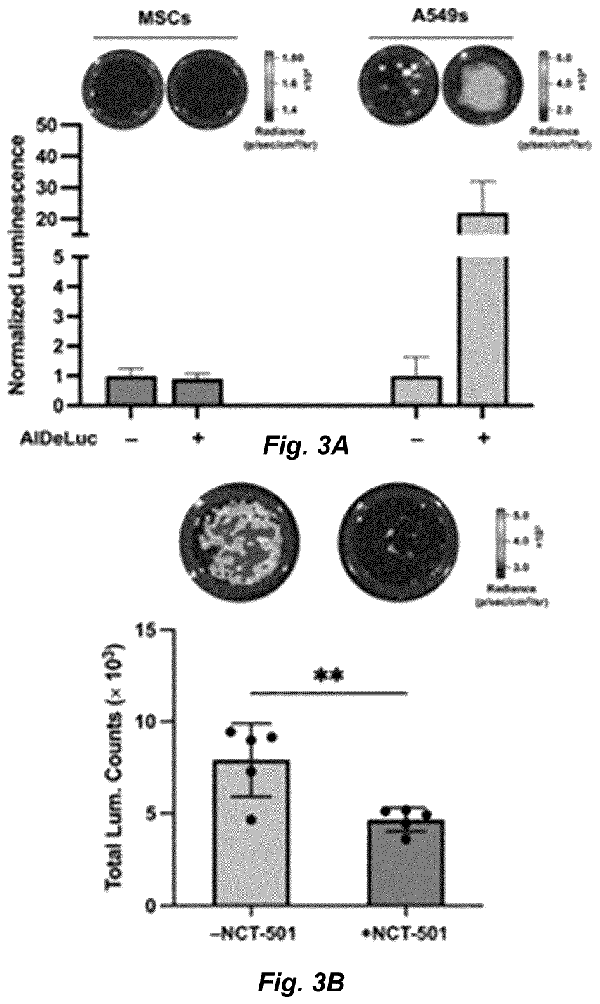

Testing commenced by incubating MSCs with AlDeLuc (20 μM). After one hour, the cells were lysed, treated with luciferase, and imaged. Notably, we devised this procedure where luciferase was supplemented because the MSCs we obtained are genetically unmodified. Moreover, we wanted to allow for any oxidized N,N-diethyl luciferin substrate to accumulate for the highest possible signal. The bioluminescence of cells treated with AlDeLuc was indistinguishable from blank controls indicating that probe activation had not occurred in the MSCs tested (FIG. 3a).

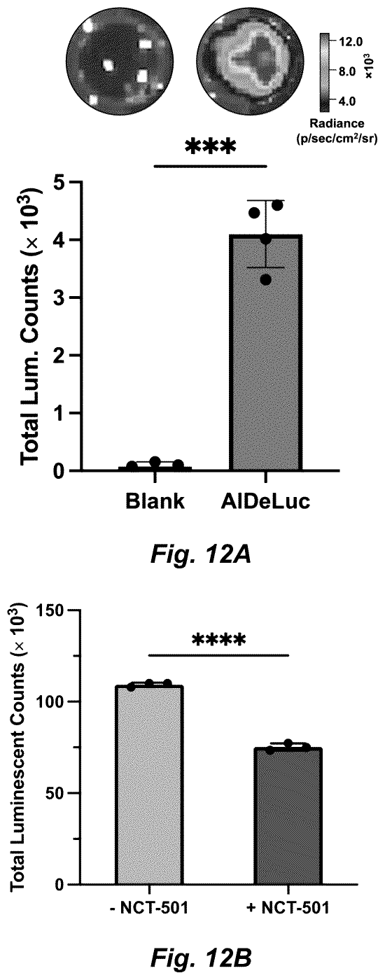

After evaluating AlDeLuc in MSCs, we further chose to use A549 human lung carcinoma cells to evaluate its performance in cancer cells. A549 cells are ideal for this application owing to their higher expression levels of ALDH1A1 compared to other cell types, with concentrations typically in the nanomolar (nM) range. When we repeated the experiment using only half the number of A549 cells relative to the number of MSCs (˜1.25×10⁵ cells), we observed that the light emission was 22.1-fold higher than the vehicle controls (FIG. 3a). By increasing the number of A549 cells, the bioluminescence intensity increased in a cell number-dependent manner (e.g., 56-fold for 3.6×10⁶ A549 cells) (FIG. 12A). We attribute the stark difference of AlDeLuc behavior in MSCs and A549 cells due to the presence of our logic-gate design featuring the acetal trigger. Next, to confirm activation of AlDeLuc was due to ALDH1A1 activity, we utilized NCT-501, a selective inhibitor of human ALDH1A1. We observed a marked reduction of 1.7-fold in bioluminescence with respect to vehicle controls (FIG. 3b). Comparable results from A549 cells were obtained when this was repeated using luciferase-expressing A549 cells (FIG. 12B). This critical finding demonstrates that AlDeLuc is an effective reporter of ALDH1A1 activity in a cellular context.

Assessment of AlDeLuc in 4T1 Murine Breast Cancer Cells. In addition to our investigation in a human lung cancer cell line, we performed additional experiments using cultured 4T1 murine mammary carcinoma cells. Recently, we demonstrated that a chronic inflammatory response, driven by diet-induced nitric oxide (NO) production, increases tumor aggressiveness, growth, and potentially sternness characteristics in 4T1 tumors. Given the pivotal role of ALDH1A1 activity in these processes, we posited that altering NO levels may affect the activity of this key enzyme. With the AlDeLuc technology in hand, we were in position to determine whether ALDH1A1 (and hence stemness) is indeed altered in the presence of NO.

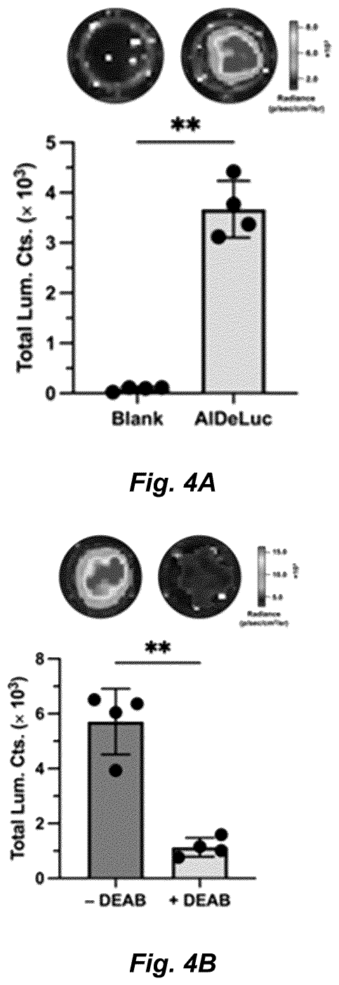

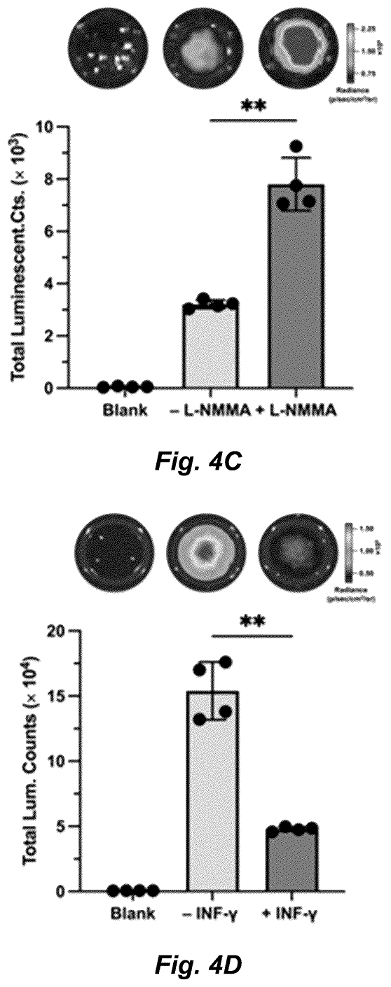

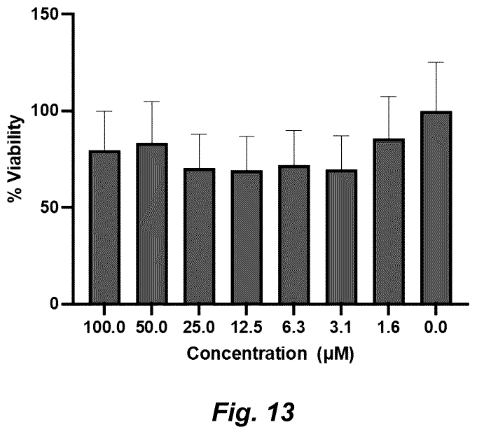

First, we showed via MTT analysis that AlDeLuc was nontoxic up to the highest concentration tested (100 μM) (FIG. 13). Next, we treated 4T1 cells with AlDeLuc to measure ALDH1A1 activity. Consistent with previous reports documenting high ALDH1A1 expression levels in this cell line, we observed a significant 42-fold increase in bioluminescence as compared to blank controls (FIG. 4a). To investigate whether this was attributable to ALDH1A1 activity, we employed diethylaminobenzaldehyde (DEAB), a competitive and reversible ALDH inhibitor, in lieu of the detrimental impact of DSF on 4T1 cell viability. Relative to uninhibited cells, DEAB treatment resulted in a diminished bioluminescent signal of approximately 5-fold (FIG. 4b). These findings collectively affirm the efficacy of AlDeLuc as a sensitive and reliable reporter of ALDH1A1 activity in 4T1 murine cells, setting the stage for its use in exploring potential crosstalk with the inflammatory response.

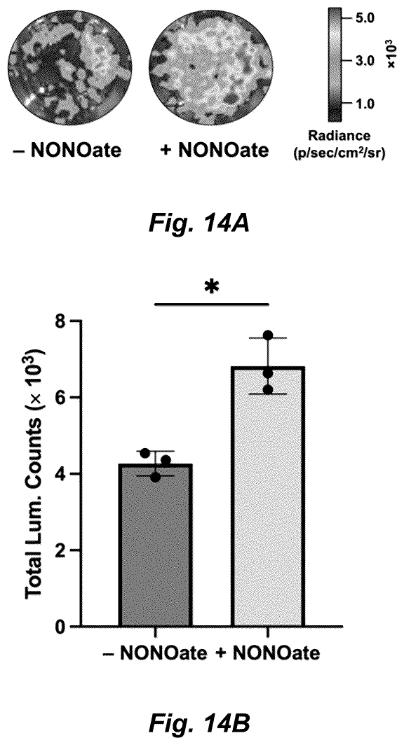

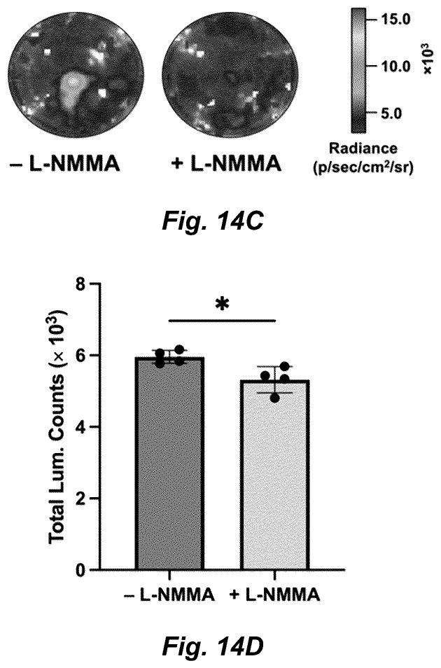

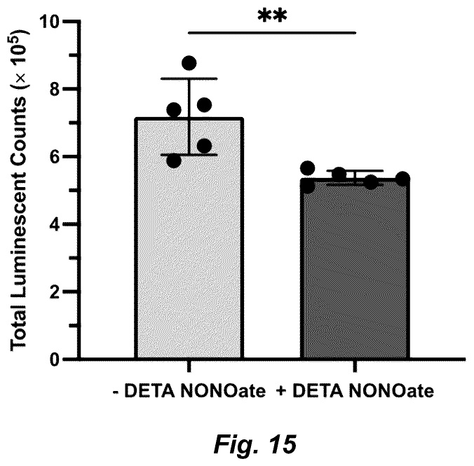

As mentioned, we previously discovered that treatment of 4T1 cells with DEA NONOate (50 μM), an NO donor, or L-NMMA (1 mM), a non-specific inhibitor of NOS, were effective at increasing or decreasing intracellular levels of NO, respectively (FIG. 14). In this study, when AlDeLuc was applied to L-NMMA-treated cells the corresponding bioluminescence was found to be 2.4-fold higher compared to vehicle-treated cells. This difference in signal indicates that attenuating NO levels has an effect of elevating ALDH1A1 activity (FIG. 4c). Based on this finding, we posited that stimulation of NO production should therefore have the opposite effect. To investigate this hypothesis, we first delivered NO exogenously using DETA NONOate, a NO donor exhibiting a long half-life (t_{1/2}=20 hours) to mimic chronic release of NO in the TME. When AlDeluc was subsequently employed to image ALDH1A1 activity, we found the bioluminescence was decreased by 1.3-fold (FIG. 15). Next, we exposed 4T1 cells to interferon gamma (IFN-γ) which has been shown to stimulate NO production via inducible nitric oxide synthase (iNOS). Similar to what we observed for NO supplementation, the bioluminescence signal of IFN-γ-treated cells was 3.2-fold lower relative to vehicle-treated control cells (FIG. 4d). While these results underscore an interaction between NO levels and ALDH1A1 activity, it is crucial to acknowledge that cellular models cannot fully recapitulate the TME. Additionally, it is important to recognize that the timing and dosage of NO production (acute versus chronic) can have vastly different effects on cancer cell properties.

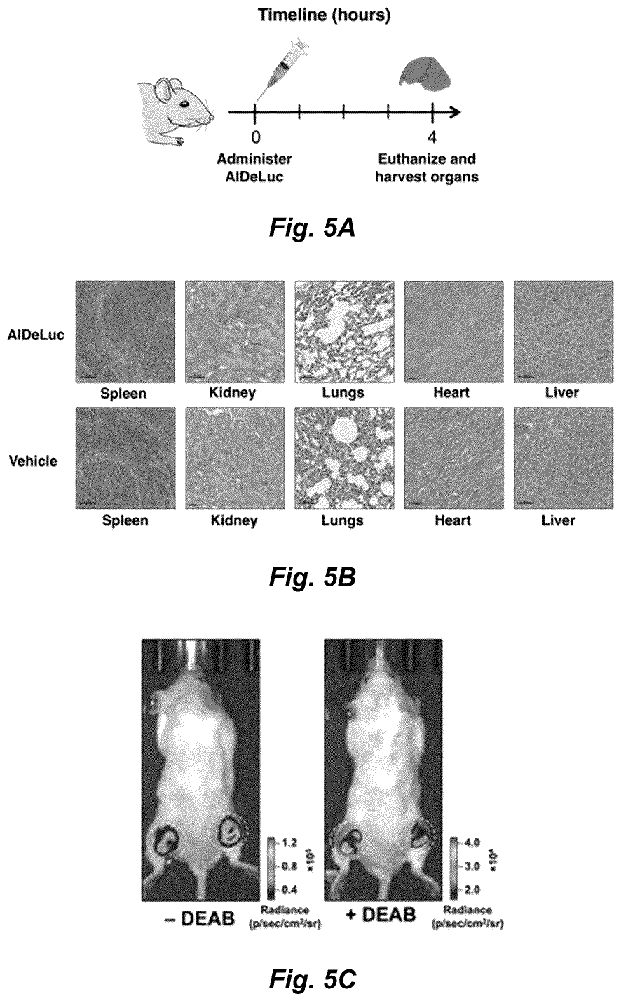

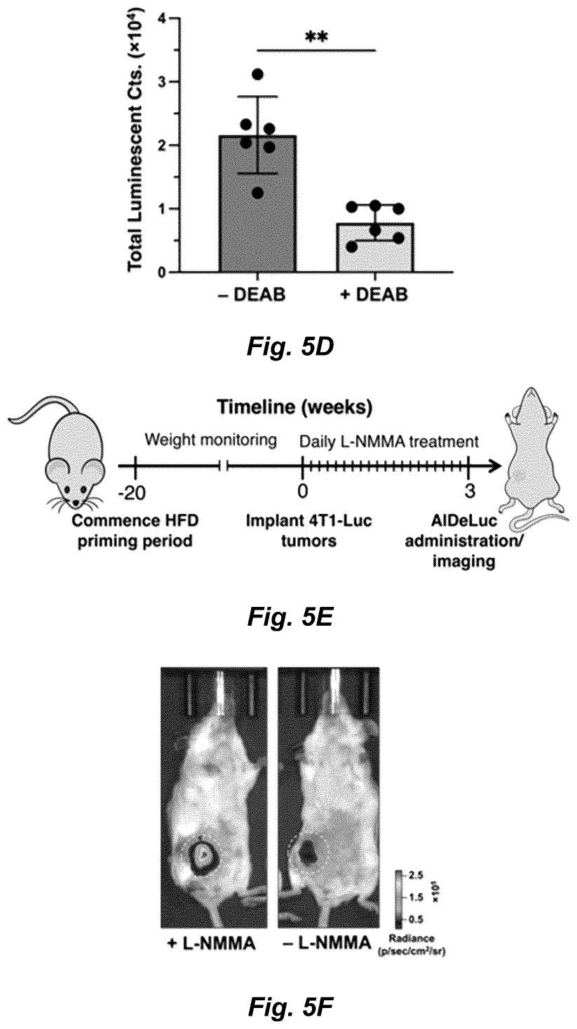

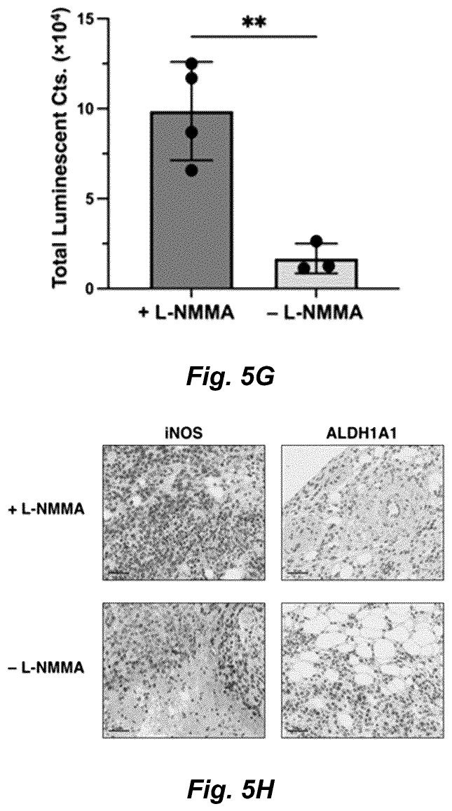

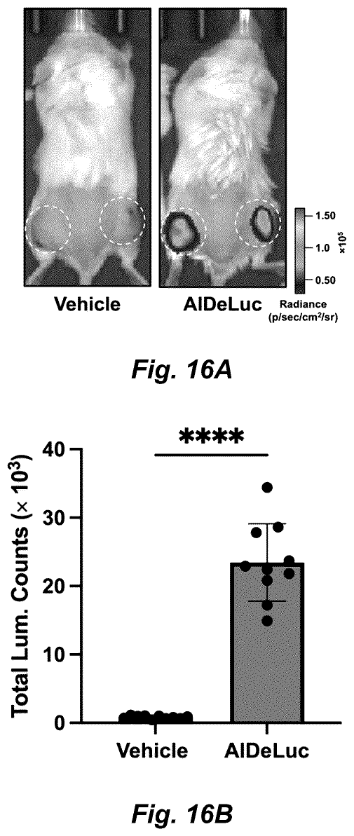

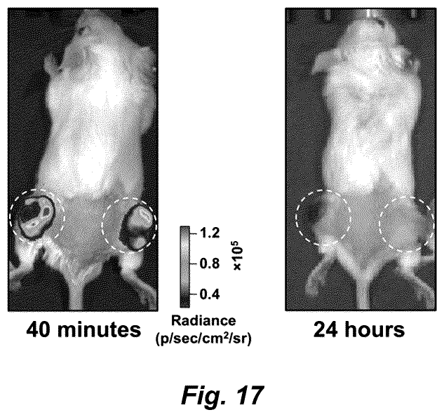

In Vivo Evaluation of AlDeLuc in 4T1 Murine Breast Tumors. Following these pivotal cellular experiments, we next evaluated AlDeLuc within an in vivo setting (FIG. 16). To establish whether AlDeLuc exhibits a favorable safety profile, we administered AlDeLuc systemically at dose of 0.55 mg/kg. After four hours, the animals were sacrificed and their vital organs including the spleen, kidneys, lungs, heart, and liver were excised for hematoxylin and eosin (H&E) staining (FIG. 5a). Compared to control animals that received a vehicle, the stains were indistinguishable, demonstrating AlDeLuc is biocompatible (FIG. 5b). Subsequently, BALB/c mice (˜5 weeks old) were inoculated on their flanks with 4T1-Luc cells (1×10⁵) to establish heterotopic breast tumors. After approximately three weeks, the average tumor volume reached ˜250 mm³. AlDeLuc was then administered systemically, and the bioluminescence output was monitored for 24 hours by delineating regions of interest (ROIs) around each tumor and summing the total luminescent counts for each mouse. AlDeLuc activation was rapid, with a detectable signal from the first scan that plateaued after 40 minutes and was absent at the 24-hour mark, indicating complete clearance (FIG. 17). This rapid clearance was crucial as it permitted subsequent experiments to confirm ALDH1A1 involvement with the same subjects, thereby minimizing variability as opposed to using a separate cohort of tumor-bearing animals. Specifically, two days later, mice were co-treated with DEAB and AlDeLuc via retroorbital injection. A significant attenuation of total luminescent counts was observed in animals co-treated with DEAB (0.78±0.28×10⁴) compared to those administered AlDeLuc alone (2.18±0.60×10⁴) (FIGS. 5c and 5d). This represents an 2.8-fold difference and demonstrate AlDeLuc's successful hydrolysis in the acidic TME and its capability to detect ALDH1A1 activity in vivo.

Diet-Induced Inflammation Modulates ALDH1A1 Activity in a Murine Model of Breast Cancer. Finally, to examine how NO influences ALDH1A1 activity in vivo we placed BALB/c mice (female, 5-7 weeks old) on a high-fat diet for 40 weeks to induce an inflammatory response through elevated NO production. This period consists of a ˜20-week priming period prior to inoculation of the animals with 4T1-Luc cells at the mammary fat pad. At this point, the mice were randomized into two groups. The treatment group was administered L-NMMA (50 mg/kg), whereas the control group received water via oral gavage for 20 days. The day after the final treatment, mice from both conditions were treated systemically with AlDeLuc, and the bioluminescence was monitored (FIG. 5e). The group administered the NOS inhibitor had a 5.9-fold enhancement in total luminescent counts as compared to the control group (9.9±2.7×10⁴ and 1.7±0.8×10⁴ respectively) (FIGS. 5f and 5g). At the completion of the diet study, animals were sacrificed, and their tumors were excised for further immunohistochemical analysis. Specifically, we determined that iNOS expression in tumors treated with L-NMMA was reduced. Not only does this result indicate, iNOS inhibition was successful but that the total amount of protein had also decreased. On the other hand, we observed the opposite trend for ALDH1A1 expression (FIG. 5h). That is, reducing the levels of NO has the effect of increasing ALDH1A1 activity (as determined with AlDeLuc), as well as overall protein expression levels (FIG. 5h). Collectively, our results provide compelling evidence that an excessive inflammatory TME modulates ALDH1A1 activity, and hence sternness properties of cancer cells.

Conclusion. Understanding cancer progression requires deep insight into the critical role of CSCs and their dynamic interplay with the TME. In this work, we have developed a bioluminescent logic-gated probe specifically for in vivo detection of CSCs via ALDH1A1 activity. Bioluminescence was chosen over other imaging modalities for its low background and high sensitivity, which are particularly advantageous for detecting rare cell populations such as CSCs. To prevent false positives due to ALDH1A1 activity at distal sites, such as within healthy tissues, we engineered a new ABS trigger that requires a two-step activation mechanism: first by the acidic TME and subsequently by ALDH1A1, to produce the active amino luciferin substrate. While several bioluminescent probes that employ dual activation exist, AlDeLuc represents the first probe of its kind that has been optimized to consider the activity of both targets, specifically fine tuning of the acid-responsive group for stability in circulation and robust unmasking in the TME, as well as the aldehyde substrate for exceptional ALDH1A1 isoform specificity. Existing probes lack these key features and therefore, are only suitable for in vitro testing under stringent conditions. Of note, we also demonstrated that the acetal moiety of AlDeLuc did not exhibit cross reactivity with CYP450s as was reported for similar chemical structures.

In our cellular studies, AlDeLuc revealed a previously uncharted interaction between ALDH1A1 activity and NO modulators like IFN-γ and L-NMMA, prompting us to explore this dynamic in vivo with an intact TME. As previously reported, we fed mice a high-fat diet to induce obesity, leading to visceral fat accumulation. Fat deposits in organs such as the liver and mammary fat pads are known to promote a chronic inflammatory state through NO overproduction. Subsequent AlDeLuc imaging uncovered a novel link between the TME's inflammatory milieu and CSC properties. This critical insight suggests that modulating NO levels could be a strategic approach to altering the stemness and survival of CSCs within tumors. Ongoing research into how NO affects the molecular function of ALDH1A1 may pave the way for new methods to target CSCs and is actively being pursued in our laboratory.

In closing, detecting nuanced cell populations and dynamic interactions like the crosstalk between NO and ALDH1A1 would not have been feasible without AlDeLuc. We anticipate that the AlDeLuc technology will prove invaluable in monitoring stem cell populations during chemotherapeutic interventions or assessing the therapeutic impact of ALDH1A1-targeted treatments.

### Pharmaceutical Formulations.

The compounds described herein can be used to prepare therapeutic pharmaceutical compositions, for example, by combining the compounds with a pharmaceutically acceptable diluent, excipient, or carrier. The compounds may be added to a carrier in the form of a salt or solvate. For example, in cases where compounds are sufficiently basic or acidic to form stable nontoxic acid or base salts, administration of the compounds as salts may be appropriate. Examples of pharmaceutically acceptable salts are organic acid addition salts formed with acids that form a physiologically acceptable anion, for example, tosylate, methanesulfonate, acetate, citrate, malonate, tartrate, succinate, benzoate, ascorbate, α-ketoglutarate, and β-glycerophosphate. Suitable inorganic salts may also be formed, including hydrochloride, halide, sulfate, nitrate, bicarbonate, and carbonate salts.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium or lithium) or alkaline earth metal (for example, calcium) salts of carboxylic acids can also be prepared by analogous methods.

The compounds of the formulas described herein can be formulated as pharmaceutical compositions and administered to a mammalian host, such as a human patient, in a variety of forms. The forms can be specifically adapted to a chosen route of administration, e.g., oral or parenteral administration, by intravenous, intramuscular, topical or subcutaneous routes.

The compounds described herein may be systemically administered in combination with a pharmaceutically acceptable vehicle, such as an inert diluent or an assimilable edible carrier. For oral administration, compounds can be enclosed in hard or soft shell gelatin capsules, compressed into tablets, or incorporated directly into the food of a patient's diet. Compounds may also be combined with one or more excipients and used in the form of ingestible tablets, buccal tablets, troches, capsules, elixirs, suspensions, syrups, wafers, and the like. Such compositions and preparations typically contain at least 0.1% of active compound. The percentage of the compositions and preparations can vary and may conveniently be from about 0.5% to about 60%, about 1% to about 25%, or about 2% to about 10%, of the weight of a given unit dosage form. The amount of active compound in such therapeutically useful compositions can be such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose or aspartame; or a flavoring agent such as peppermint, oil of wintergreen, or cherry flavoring, may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

The active compound may be administered intravenously or intraperitoneally by infusion or injection. Solutions of the active compound or its salts can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising the active ingredient adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and/or antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by agents delaying absorption, for example, aluminum monostearate and/or gelatin.

Sterile injectable solutions can be prepared by incorporating the active compound in the required amount in the appropriate solvent with various other ingredients enumerated above, as required, optionally followed by filter sterilization. In the case of sterile powders for the preparation of sterile injectable solutions, methods of preparation can include vacuum drying and freeze drying techniques, which yield a powder of the active ingredient plus any additional desired ingredient present in the solution.

For topical administration, compounds may be applied in pure form, e.g., when they are liquids. However, it will generally be desirable to administer the active agent to the skin as a composition or formulation, for example, in combination with a dermatologically acceptable carrier, which may be a solid, a liquid, a gel, or the like.

Useful solid carriers include finely divided solids such as talc, clay, microcrystalline cellulose, silica, alumina, and the like. Useful liquid carriers include water, dimethyl sulfoxide (DMSO), alcohols, glycols, or water-alcohol/glycol blends, in which a compound can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using a pump-type or aerosol sprayer.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user.

Examples of dermatological compositions for delivering active agents to the skin are known to the art; for example, see U.S. Pat. No. 4,992,478 (Geria), U.S. Pat. No. 4,820,508 (Wortzman), U.S. Pat. No. 4,608,392 (Jacquet et al.), and U.S. Pat. No. 4,559,157 (Smith et al.). Such dermatological compositions can be used in combinations with the compounds described herein where an ingredient of such compositions can optionally be replaced by a compound described herein, or a compound described herein can be added to the composition.

Useful dosages of the compounds described herein can be determined by comparing their in vitro activity, and in vivo activity in animal models. Methods for the extrapolation of effective dosages in mice, and other animals, to humans are known to the art; for example, see U.S. Pat. No. 4,938,949 (Borch et al.). The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

The compound can be conveniently administered in a unit dosage form, for example, containing 5 to 1000 mg/m², conveniently 10 to 750 mg/m², most conveniently, 50 to 500 mg/m² of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. Experimental Details

Materials. Materials were purchased from commercial vendors and used without further purification. All deuterated solvents were purchased from Cambridge Isotope Laboratories. Acetone, dichloromethane, dimethyl sulfoxide, glacial acetic acid, phosphate saline buffer (PBS) (Corning), and Matrigel were purchased from Corning. Nuncon Delta Surface 96-well plate, sodium chloride, and sodium hydroxide were purchased from Thermo Fisher Scientific. Sodium hypochlorite (14.5% available chlorine in water) was purchased from Alfa Aesar. Agarose LE (Molecular Biology Grade) was purchased from Gold Biotechnology. Acetonitrile, anhydrous methanol, concentrated hydrochloric acid, hydrogen peroxide (30% v/v) and sodium hydroxide were purchased from Macron Fine Chemicals. 2-chlorobenzothiazole, D-cysteine hydrochloride, 1-hydroxyl-7-azabenzotriazole (HOAt), tris(2-carboxyethyl)phosphine hydrochloride (TCEP·HCl) was purchased from Ambeed Inc. Cesium carbonate, citric acid, N,N-diisopropylethylamine hexafluorophosphate azabenzotriazole tetramethyl uronium (HATU), di-tert-butyl dicarbonate, N,O-dimethylhydroxylamine hydrochloride, dimethoxy propane, 4 M HCl in dioxanes, iron powder, NADH, potassium cyanide, potassium nitrate, magnesium sulfate, sodium carbonate, and sodium triacetoxyborohydride were purchased from Oakwood Chemicals. Acetaldehyde, β-galactosidase from Escherichia coli, anhydrous dimethylformamide, anhydrous methanol, anhydrous pyridine, anhydrous tetrahydrofuran, hydrogen sulfide, MTT reagent (3-(4,5-Dimethylthiazol-2-yl)-2,5-diphenyl tetrazolium bromide), reduced glutathione, sodium hypochlorite, adenosine 5′-triphosphate magnesium salt, and tert-Butyl hydroperoxide solution 70% in H₂O were purchased from Millipore-Sigma Aldrich. Beta-nicotinamide adenine dinucleotide was purchased from Combi Blocks. Ethanol 200 proof was purchased from Decon Labs Inc. Isopropyl alcohol and methanol were purchased from Macron Fine Chemicals. ACS grade hydrochloric acid, ACS grade glacial acetic acid, ACS grade hydrosulfuric acid, and dimethyl sulfoxide were purchased from VWR chemical. Lithium aluminum hydride and dichloroethane were purchased from Alfa Aesar. Anhydrous acetone, hexanes, ethyl acetate, dichloromethane, diethyl ether were purchased from Fisher Scientific. DEA-NONOate, Luciferase, and L-NMMA was purchased from Cayman Chemical. 4T1, 4T1-Luc, and A549 cells were purchased from ATCC. BALB/c mice were purchased from the Jackson Laboratory.

Instruments and Software. 1H and 13C NMR spectra were acquired on the Carver B500 spectrometer. The following abbreviations were used to describe coupling constants: singlet (s), doublet (d), triplet (t), quartet (q), or multiplet (m). Spectra were visualized and analyzed using MestReNova (version 10.0) and referenced to trace non-deuterated solvent. High-resolution mass spectra were acquired on a Waters Q-TOF Ultima ESI mass spectrometer or a Waters Synapt G2-Si ESI/LC-MS spectrometer. Ultraviolet-visible spectroscopy was performed on a Cary 60. Ultraviolet-visible spectroscopy was performed with a micro fluorescence quartz cuvette (Science Outlet). Cells were visualized on an EVOS FL epifluorescence microscope and a Countess II Automated Cell Counter (Invitrogen). SpectraMax M2 plate reader (Molecular Devices) was used for cell viability assays and for AlDeLuc bioluminescent emission spectra collection. Data was analyzed using Microsoft Excel. Figures were generated using Prism.

### Synthetic Methods.

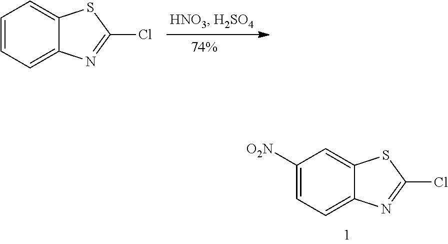 [SMILES: Clc1nc2ccccc2s1 ; O=[N+]([O-])c1ccc2nc(Cl)sc2c1]

2-Chloro-6-nitrobenzo[d]thiazole (1). H₂SO₄ (51.2 mL) was cooled to 0° C. in an ice-bath. 2-Chlorobenzo[d]thiazole (6 mL, 45.06 mmol, 1 equiv.) was then added dropwise to the reaction. While maintaining the temperature at 0° C., KNO₃ (5.01 g, 49.57 mmol, 1.1 equiv.) was added portion-wise over 30 minutes. After stirring for an additional 30 minutes, the reaction mixture was warmed to room temperature and stirred overnight. The reaction was quenched via pouring over ice.

The solid was then filtered, washed with ice-cold deionized water, and recrystallized in ethanol to give 1 as light-yellow crystals in 74% yield (7.32 g, 33.34 mmol). ¹H NMR (500 MHz, CDCl₃) δ 8.68 (d, J=2.2 Hz, 1H), 8.31 (dd, J=9.0, 2.3 Hz, 1H), 8.01 (d, J=8.9 Hz, 1H). ¹³C NMR (126 MHz, CDCl₃) δ 158.81, 154.78, 145.44, 136.47, 123.37, 122.27, 117.71.

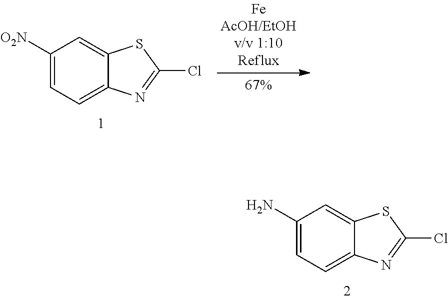 [SMILES: O=[N+]([O-])c1ccc2nc(Cl)sc2c1 ; Nc1ccc2nc(Cl)sc2c1]

2-Chloro[d]thiazole-6-amine (2). Iron powder (4.29 g, 76.82 mmol, 4 equiv.) was added to a solution of 1 (4.12 g, 19.21 mmol, 1 equiv.) in 1:10 v/v acetic acid:ethanol (0.1 μM). After stirring at reflux for two hours, the reaction mixture was cooled to room temperature, diluted with ethanol, and filtered over celite. After the volume of the filtrate was reduced to ˜50% under reduced pressure, the remaining liquid was neutralized and extracted using ethyl acetate (×3). The combined organic layers were washed with brine, dried over Na₂SO₄, and concentrated to dryness. The solid residue was purified via recrystallization in ethanol to afford 2 as light purple crystals in 67% yield (2.37 g, 12.86 mmol). ¹H NMR (500 MHz, CDCl₃) δ 7.70 (d, J=8.7 Hz, 1H), 6.99 (d, J=2.3 Hz, 1H), 3.86 (s, 2H). ¹³C NMR (126 MHz, CDCl3) δ 148.47, 144.94, 144.13, 137.83, 123.39, 115.70, 105.16.

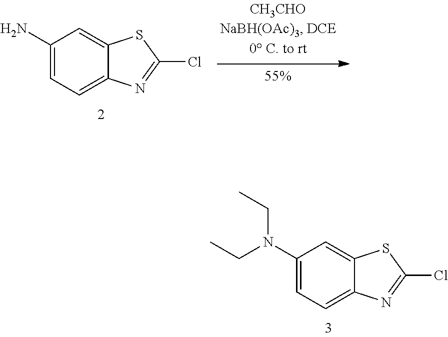

2-Chloro-N,N-diethylbenzo[d]thiazole-6-amine (3). To a solution of 2 (1.33 g, 7.19 mmol, 1 equiv.) in dichloroethane (65.40 mL, 0.11 μM), acetaldehyde (2.43 mL, 43.16 mmol, 6 equiv.) was added. Immediately after addition, sodium triacetoxyborohydride (3.81 g, 17.98 mmol, 2.5 equiv.) was added and stirred at room temperature for two hours. The reaction was quenched with methanol and the solvent was concentrated. The residue was redissolved in ethyl acetate, washed with water (×2), dried over sodium sulfate, and concentrated. The crude was then purified via silica gel column chromatography (eluent: 3:97 v/v EtOAc:Hexanes) to afford 3 as a brown oil in 55% yield (0.9515 g, 3.95 mmol). ¹H NMR (500 MHz, CDCl₃) δ 7.71 (d, J=9.1 Hz, 1H), 6.90 (d, J=2.6 Hz, 1H), 6.84 (dd, J=9.1, 2.6 Hz, 1H), 3.39 (q, J=7.1 Hz, 5H), 1.19 (t, J=7.1 Hz, 7H). ¹³C NMR (126 MHz, CDCl₃) δ 146.83, 146.42, 141.81, 138.60, 123.05, 112.65, 101.51, 44.85, 12.52.

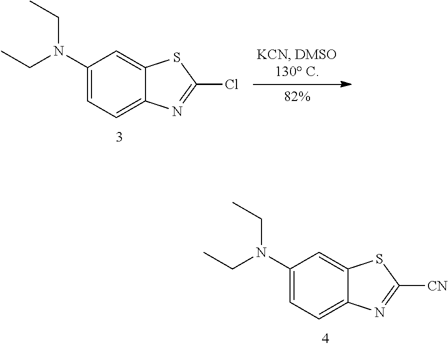 [SMILES: CCN(CC)c1ccc2nc(Cl)sc2c1 ; CCN(CC)c1ccc2nc(C#N)sc2c1]

6-(Diethylamino)benzo[d]thiazole-2-carbonitrile (4). A round-bottom flask was charged with potassium cyanide (56.85 mg, 0.87 mmol, 2 equiv.) and treated with a solution of 3 (105.10 mg, 0.44 mmol, 1 equiv.) in DMSO (17 mL, 0.03 M). The reaction was stirred at 130° C. for three hours, cooled to room temperature, and poured onto deionized water (100 mL). The reaction was transferred to a separatory funnel, extracted with EtOAc (×3), dried with Na₂SO₄, and concentrated. The crude residue was purified by silica gel column chromatography (eluent: 7:93 v/v EtOAc:Hexanes) to afford 4 as a yellow solid in 82% yield (0.0828 g, 0.36 mmol). ¹H NMR (500 MHz, CDCl₃) δ 7.93 (d, J=9.3 Hz, 1H), 7.00 (dd, J=9.3, 2.6 Hz, 1H), 6.96 (d, J=2.6 Hz, 1H), 3.46 (q, J=7.1 Hz, 5H), 1.23 (t, J=7.1 Hz, 7H). ¹³C NMR (126 MHz, CDCl₃) δ 148.37, 143.39, 139.14, 128.87, 125.48, 114.46, 114.10, 99.93, 45.01, 12.50.

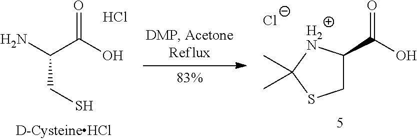

(S)-4-carboxy-2,2-dimethylthiazolidin-3-ium chloride (5). To a solution of D-cysteine hydrochloride (21.05 g, 133.56 mmol, 1 equiv.) in acetone (530 mL, 0.24 M) was added 2,2-dimethoxypropane (111 mL, 908.56 mmol, 7.16 equiv.) and refluxed for 20 hours. The reaction was cooled to 50° C., filtered, and washed with cold acetone. The white crystals were collected to give 6 in 83% yield (21.91 g, 110.85 mmol) and used without further purification. ¹H NMR (500 MHz, D₂O) δ 4.88 (t, J=8.1 Hz, 1H), 3.65 (dd, J=12.2, 8.1 Hz, 1H), 3.52 (dd, J=12.2, 8.0 Hz, 1H), 1.79 (d, J=6.9 Hz, 6H). ¹³C NMR (126 MHz, D₂O) δ 169.81, 72.87, 62.15, 31.59, 27.65, 26.61.

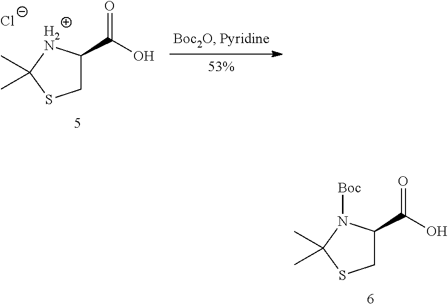

(S)-3-(tert-butoxycarbonyl)-2,2-dimethylthiazolidine-4-carboxylic acid (6). Di-tert-butyl dicarbonate (11.14 g, 51.04 mmol, 1.1 equiv.) was added to a solution of 5 (7.48 g, 46.40 mmol, 1 equiv.) in pyridine (45 mL, 1.04 μM), flushed with nitrogen, and stirred at room temperature for 2.5 days. The reaction was diluted with toluene, transferred to a separatory funnel, and extracted with ice-cold 2 M sodium hydroxide (×3). The aqueous fraction was washed with toluene (×3), Hexanes (×1), and acidified with aqueous citric acid to pH 3.0. After acidification, the aqueous solution was extracted with dichloromethane (×4). The organic fraction was washed with brine, dried with Na₂SO₄, and concentrated. The resulting crude residue was recrystallized in Hexanes to afford the product as an off-white solid in 53% yield (7.18 g, 29.87 mmol). ¹H NMR (500 MHz, CDCl₃) δ 11.49-10.76 (m, 1H), 5.11-4.48 (m, 1H), 3.34-3.02 (m, 2H), 1.83-1.66 (m, 6H), 1.40 (d, J=43.5 Hz, 9H). ¹³C NMR (126 MHz, CDCl₃) δ 177.28, 175.54, 153.74, 151.64, 81.89, 80.97, 71.90, 70.37, 65.95, 65.26, 30.73, 30.32, 29.74, 29.59, 28.69, 28.39, 28.28, 27.95.

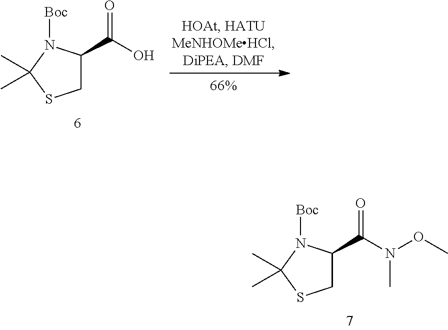

tert-Butyl (S)-4-methoxy(methyl)carbamoyl)-2,2-dimethylthiazolidine-3-carboxylate (7). To a solution of 6 (3.00 g, 11.48 mmol, 1.0 equiv.) in DMF (15.3 mL, 0.75 μM), was sequentially treated with N,N-diisopropylethylamine (4 mL, 22.96 mmol, 2.0 equiv.), hexafluorophosphate azabenzotriazole tetramethyl uronium (HATU) (5.50 g, 14.46 mmol, 1.3 equiv.), 1-hydroxyl-7-azabenzotriazole (1.77 g, 12.97 mmol, 1.1 equiv.), and N,O-dimethylhydroxylamine hydrochloride (2.24 g, 22.96 mmol, 2.0 equiv.). The resulting mixture was stirred at room temperature for 16 hours and then diluted with deionized water. The aqueous solution was extracted with EtOAc (×3), washed with 30% citric acid (×2), saturated NaHCO₃(×2), and brine (×1). It was then dried with Na₂SO₄ and concentrated. The crude residue was purified via silica gel column chromatography (eluent: 2:3 v/v EtOAc:Hexanes) to afford the product as an off-white solid in 66% yield (2.30 g, 7.58 mmol). ¹H NMR (500 MHz, CDCl₃) δ 5.11 (d, J=67.1 Hz, 1H), 3.75 (d, J=23.1 Hz, 3H), 3.33 (dd, J=13.5, 7.0 Hz, 1H), 3.21 (s, 3H), 3.01-2.94 (m, 1H), 1.89 (d, J=13.2 Hz, 6H), 1.45 (d, J=41.1 Hz, 9H). ¹³C NMR (126 MHz, CDCl₃) δ 171.61, 171.14, 170.74, 152.88, 151.64, 80.72, 80.30, 72.07, 70.41, 64.77, 63.97, 61.30, 61.19, 60.39, 32.56, 30.87, 30.15, 29.83, 29.32, 28.64, 28.48, 28.42, 27.82, 21.06, 14.20.

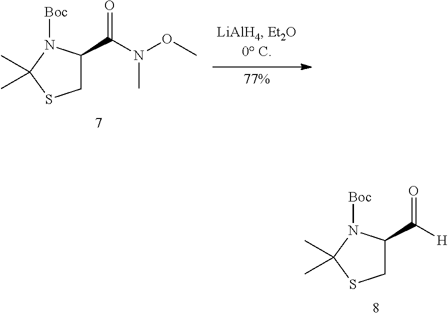

tert-Butyl (S)-4-formyl-2,2-dimethylthiazolidine-3-carboxylate (8). To a solution of 8 (500 mg, 1.64 mmol, 1.0 equiv.) in diethyl ether (5.7 mL, 0.29 M) was cooled to 0° C. In one portion, LiAlH₄ (62.34 mg, 1.64 mmol, 1.0 equiv.) was added and the resulting suspension was stirred at the same temperature for 30 minutes. While on ice, KHSO₄ (30% w/v in deionized water) was added and stirred for an additional 20 minutes. The solids were filtered and rinsed with diethyl ether. The filtrate was washed with 0.1 M HCl (×1), 10% NaHCO₃(×1), brine (×2), dried with Na₂SO₄ and concentrated. The crude residue was purified with silica gel column chromatography (eluent: 1:4 v/v EtOAc:Hexanes) to give the product as an off-white solid in 77% yield (0.3103 g, 1.26 mmol). ¹H NMR (500 MHz, CDCl₃) δ 9.58 (s, 1H), 4.62 (d, J=90.0 Hz, 1H), 3.15 (d, J=19.2 Hz, 2H), 2.11-1.66 (m, 6H), 1.47 (d, J=42.9 Hz, 9H). ¹³C NMR (126 MHz, CDCl₃) δ 200.54, 200.26, 153.47, 151.73, 81.60, 81.30, 71.89, 71.59, 70.75, 70.14, 30.36, 29.37, 29.18, 28.46, 28.36, 28.33, 27.66.

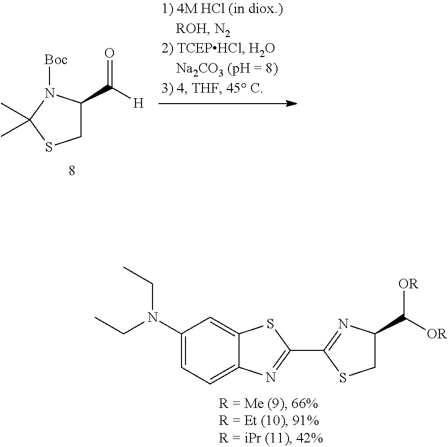

(S)-2-(4-(dimethoxymethyl)-4,5-dihydrothiazol-2-yl)-N,N-diethylbenzo[d]thiazol-6-amine (9, AlDeLuc). To a round-bottom flask containing 8 (200 mg, 0.82 mmol, 1.0 equiv.) dissolved in degassed MeOH (1.63 mL, 0.5 μM), was added 4 M hydrochloric acid in dioxanes (2.04 mL, 8.15 mmol, 10.0 equiv.). The resulting solution was stirred at room temperature under nitrogen for 16 hours. The following day, the solvent was removed by vigorously bubbling nitrogen into the reaction. The resultant oil was then treated with tris(2-carboxyethyl)phosphine hydrochloride (TCEP·HCl) (0.3973 g, 1.39 mmol, 1.70 equiv.) dissolved in degassed deionized water (1.33 mL, 1.04 M). After 10 minutes, the mixture was basified with degassed saturated Cs₂CO₃ in DMF. Compound 4 (0.2074 g, 0.90 mmol, 1.10 equiv.) was suspended in degassed tetrahydrofuran (1.34 mL, 0.67 M) and immediately added to the reaction vessel and heated to 50° C. for 1.5 days. The reaction was cooled to room temperature, diluted with water, and extracted with EtOAc (×3). The organic fraction was washed with brine (×1), dried with Na₂SO₄, and concentrated. The crude oil was purified by silica gel column chromatography (eluent: 2:8 v/v EtOAc:Hexanes) to afford a dark orange oil in 66% yield (0.2118 g, 0.54 mmol). ¹H NMR (500 MHz, CDCl₃) δ 7.91 (d, J=9.3 Hz, 1H), 6.98 (d, J=2.6 Hz, 1H), 6.91 (dd, J=9.3, 2.6 Hz, 1H), 4.87 (td, J=8.9, 5.1 Hz, 1H), 4.63 (d, J=5.1 Hz, 1H), 3.57-3.38 (m, 12H), 1.22 (t, J=7.1 Hz, 6H). ¹³C NMR (500 MHz, CDCl₃) δ 164.97, 155.02, 147.29, 144.53, 139.07, 124.94, 113.18, 105.56, 105.52, 101.08, 80.10, 56.05, 55.50, 44.88, 33.01, 12.61. HRMS [M+H]⁺ calculated mass for C₁₇H₂₃N₃O₂S₂=366.1305, found=366.4583.

(S)-2-(4-(diethoxymethyl)-4,5-dihydrothiazol-2-yl)-N,N-diethylbenzo[d]thiazol-6-amine (10). To a round-bottom flask containing 8 (200 mg, 0.82 mmol, 1.0 equiv.) dissolved in degassed EtOH (1.63 mL, 0.5 μM), was added 4 M hydrochloric acid in dioxanes (2.04 mL, 8.15 mmol, 10.0 equiv.) and stirred at room temperature under nitrogen for 16 hours. The following day, the solvent was removed by vigorously bubbling nitrogen into the reaction. The resultant oil was then treated with tris(2-carboxyethyl)phosphine hydrochloride (TCEP·HCl) (0.3973 g, 1.39 mmol, 1.70 equiv.) dissolved in degassed deionized water (1.33 mL, 1.04 M). After 10 minutes, the mixture was basified with degassed saturated Cs₂CO₃ in DMF. Compound 4 (0.2074 g, 0.90 mmol, 1.10 equiv.) was suspended in degassed tetrahydrofuran (1.34 mL, 0.67 M) and was immediately added to the reaction vessel and heated to 50° C. for 1.5 days. The reaction was cooled to room temperature, diluted with water, and extracted with EtOAc (×3). The organic fraction was washed with brine (×1), dried with Na₂SO₄, and concentrated. The crude oil was purified by silica gel column chromatography (eluent: 2:8 v/v EtOAc:Hexanes) to afford a dark orange oil in 91% yield (0.2920 g, 0.75 mmol). ¹H NMR (500 MHz, CDCl₃) δ 7.91 (d, J=9.2 Hz, 1H), 6.98 (d, J=2.6 Hz, 1H), 6.91 (dd, J=9.2, 2.6 Hz, 1H), 4.87 (ddd, J=9.2, 8.2, 4.6 Hz, 1H), 4.79 (d, J=4.7 Hz, 1H), 3.85-3.73 (m, 2H), 3.73-3.53 (m, 3H), 3.50-3.38 (m, 5H), 1.28 (t, J=7.1 Hz, 3H), 1.20 (dt, J=18.4, 7.0 Hz, 9H). ¹³C NMR (500 MHz, CDCl₃) δ 164.73, 155.20, 147.27, 144.56, 139.03, 124.94, 113.15, 103.24, 101.09, 80.79, 64.51, 63.60, 44.88, 32.98, 15.44, 15.33, 12.61. HRMS [M+H]⁺ calculated mass for C₁₉H₂₈N₃O₂S₂=394.1617, found=394.5533.

(S)-2-(4-(diisopropoxymethyl)-4,5-dihydrothiazol-2-yl)-N,N-diethylbenzo[d]thiazol-6-amine (11). To a round-bottom flask containing 8 (200 mg, 0.82 mmol, 1.0 equiv.) dissolved in degassed iPrOH (1.63 mL, 0.5 μM), was added 4 M hydrochloric acid in dioxanes (2.04 mL, 8.15 mmol, 10.0 equiv.) and stirred at room temperature under nitrogen for 16 hours. The following day, the solvent was removed by vigorously bubbling nitrogen into the reaction. The resultant oil was then treated with tris(2-carboxyethyl)phosphine hydrochloride (TCEP·HCl) (0.3973 g, 1.39 mmol, 1.70 equiv.) dissolved in degassed deionized water (1.33 mL, 1.04 M). After 10 minutes, the mixture was basified with degassed saturated Cs₂CO₃ in DMF. Compound 4 (0.2074 g, 0.90 mmol, 1.10 equiv.) was suspended in degassed tetrahydrofuran (1.34 mL, 0.67 M) and was immediately added to the reaction vessel and heated to 50° C. for 1.5 days. The reaction was cooled to room temperature, diluted with water, and extracted with EtOAc (×3). The organic fraction was washed with brine (×1), dried with Na₂SO₄, and concentrated. The crude oil was purified by silica gel column chromatography (eluent: 5:95 v/v EtOAc:Hexanes) to afford a dark orange oil in 42% yield (0.1444 g, 0.34 mmol). ¹H NMR (500 MHz, CDCl₃) δ 7.92 (d, J=9.1 Hz, 1H), 6.99 (d, J=2.5 Hz, 1H), 6.91 (dd, J=9.2, 2.5 Hz, 1H), 4.97 (d, J=4.0 Hz, 1H), 4.83 (td, J=8.8, 4.0 Hz, 1H), 3.94 (dp, J=27.1, 6.2 Hz, 2H), 3.61 (dd, J=11.0, 8.2 Hz, 1H), 3.43 (q, J=7.1 Hz, 5H), 1.27-1.18 (m, 15H), 1.08 (d, J=6.1 Hz, 3H). ¹³C NMR (500 MHz, CDCl₃) δ 164.48, 155.33, 147.25, 144.60, 138.98, 124.95, 113.12, 101.11, 99.60, 81.67, 70.18, 68.95, 44.88, 32.71, 23.20, 23.09, 22.59, 22.27, 12.61. HRMS [M+H]⁺ calculated mass for C₂₁H₃₂N₃O₂S₂=422.1930, found=422.6491.

Expression and Purification of ALDH Expression constructs for recombinant human ALDH1A1, ALDH1A2, ALDH1A3, and ALDH2, ALDH3A1, ALDH4A1, and ALDH5A1 were generously provided by Prof. Daria Mochly-Rosen (Stanford, Chemical and Systems Biology). E. coli BL21(DE3) cells with transformed with each of the above constructs. Colonies were selected from an agar plate containing 100 mg/mL ampicillin and grown overnight (37° C., 220 rpm) in LB media containing 100 mg/mL ampicillin. The overnight culture (10 mL) was inoculated into 1000 mL LB supplemented with 100 mg/mL ampicillin. When the O.D. reached ˜0.5, IPTG was added to a final concentration of 1 mM and incubated overnight (30° C., 200 rpm, 18 h). The cells were harvested via centrifugation (4° C., 4,000 rpm, 90 min) and the cell pellets were stored at −80° C. until purification.

The bacterial pellets were resuspended in BugBuster® Master Mix (EMD Millipore, 15 mL for every 500 mL of cell culture collected) combined with protease inhibitor cocktail (one tablet for every liter of cells collected, Pierce™ Protease Inhibitor Tablets, EDTA-free). They were then lysed according to manufacturer instructions (30 min incubation, room temp., rocking). The extract was centrifuged (4° C., 4,000 rpm, 90 mins) and the supernatant was applied to a Ni-NTA column. The column was washed by the following binding buffer: 20 mM sodium phosphate pH 7.4, 0.5 M NaCl, 20 mM imidazole. Washing continued until the OD₂₈₀ was less than 0.1 to ensure the removal of non-specific binders. Target proteins were then eluted by the wash buffer (20 mM sodium phosphate pH 7.4 0.5 M NaCl, 500 mM imidazole). Purity of eluted protein was determined to be >95% by SDS-PAGE and concentration of protein was determined by bicinchoninic acid (BCA) assay. Protein was stored with 50% glycerol at −80° C.

Cell Culture. 4T1-Luc cells were cultured in phenol red-free RPMI 1640 (ATCC) with 10% fetal bovine serum (FBS, Sigma Aldrich) and 1% penicillin/streptomycin (Corning). 4T1 cells were cultured in phenol red-free RPMI 1640 (ATCC) with 10% fetal bovine serum (FBS, Sigma Aldrich) and 1% penicillin/streptomycin (Corning). A549 cells were cultured in F-12K medium (ATCC) with 10% fetal bovine serum (FBS, Sigma Aldrich) and 1% penicillin/streptomycin (Corning).

MTT Cytotoxicity Assay. 48-well plates were seeded with 100,000 cells per well (2 mL of 50,000 cells/mL) and incubated at 37° C. with 5% CO₂ for 16 hours (˜75% confluent). Media was removed and fresh RPMI 1640 media with 10% FBS containing 100, 50 25, 12.5, 6.25, 3.13, 1.56, or 0 μM AlDeLuc (1% DMSO final v/v) was added. The media was removed after two hours and replaced with 500 μL of 18:1 mixture of FBS-free RPMI 1640 and (3-(4,5-dimethylthiazol-2-yl)-2,5-diphenyltetrazolium bromide (MT, 5 mg/mL stock in PBS). The cells were incubated for one hour at the same conditions before the media was removed. DMSO (500 μL/well) was added, and the absorbance was read at 555 nm on a microplate reader. Viability was calculated by the absorbance relative to the vehicle control. Values are reported as the mean±standard deviation (n=3).

Formulation of AlDeLuc for In Vivo Imaging. AlDeLuc was dissolved in DMSO and diluted in PBS (1:9 v/v DMSO:PBS). The solution was filtered through a 0.22 M sterile filter immediately before use. The typical injection volume was approximately 100 L at a dose of 0.55 mg/kg.

Evaluation of Acetal Response to 1 M HCl. To identify the most robust turn-on to be utilized for this study, compounds 9, 10, and 11 were tested for their ability to be deprotected under acidic conditions. Each acetal was dissolved in DMSO and subjected to equal-volume 1 M HCl and incubated at room temperature for 60 minutes. After this time, each solution was neutralized with 1 M NaOH and diluted to a concentration of 1 mM with PBS. In a black 96-well plate, PBS, NAD⁺ (2.5 mM), ALDH1A1 (0.4 μM), and either compound 9, 10 or 11 (100 μM) were added. After 5 minutes, each well was treated with ATP-MgSO₄ (45 μL, prepared by mixing 17.78 mM ATP-Mg and 35.56 mM MgSO₄ in a 1:1 ratio in PBS buffer) and luciferase (5 μL, 1 mg/mL in PBS containing 10% glycerol) to initiate bioluminescence production. Light was collected immediately after mixing, and the signal enhancement was determined relative to control wells containing no probe or enzymes. The total volume of each well was 200 μL (PBS (pH 7.4) with 0.5% DMSO). All data reported as the mean±standard deviation (n=4).

*Prior to assay, stock solutions of 9, 10, and 11 were diluted in DMSO (100 μM) was diluted and analyzed via UV-VIS-NIR spectrophotometry to ensure the concentrations were matched (up to 10% variability is considered acceptable).

Analyte Selectivity Assay for AlDeLuc Aldehyde Product. The response of the AlDeLuc aldehyde product (50 μM) toward a panel of biologically relevant reactive oxygen species (100 μM), reactive nitrogen species (100 μM), and reactive sulfur species (1 mM) were monitored using the IVIS imaging system in a 96-well plate. AlDeLuc was incubated with 1 M HCl for 1 hour, neutralized with 1 M NaOH, and diluted with HEPES buffer. The aldehyde was then incubated with each analyte for one hour at room temperature, for a final volume of 100 L, before the reaction was initiated by adding ATP-MgSO₄ and luciferase. Light was collected within 10 minutes after mixing and the signal enhancement was determined relative to wells treated with ALDH1A1. Tert-butoxide and hydroxide radicals were generated according to the reported literature. NO was generated in situ from a solution of DEA-NONOate in degassed PBS buffer. Reduced glutathione was maintained at the final concentration of 1 mM in the reaction mixture. ALDH1A1 was maintained at a final concentration of 21.82 μM. All other analytes were prepared by dilution of dissolution from commercially available sources.

ALDH Isoform Activity Determination. The activity of each isoform of ALDH was confirmed by monitoring the production of NADH at 340 nm when incubated with the most used substrate for that enzyme (i.e., benzaldehyde for ALDH3A1 and propionaldehyde for all other isoforms). Each isoform was diluted with 50 mM triethanolamine (TEA, pH 7.4) to a final concentration of 0.5 μM and placed in a 1 mL quartz cuvette. Prior to measurement, NAD⁺ was added to a final concentration of 2.5 mM and the preferred substrate was added to a final concentration of 1 mM. Absorbance spectra was taken from 300 to 500 nm every 30 seconds for 15 minutes. Activity for each enzyme was calculated from the slope of absorbance increase at 340 nm over time.

AlDeLuc Isoform Selectivity Assay. Activation of AlDeLuc was assessed using the necessary amount of enzyme that gave the activity of 0.5 nmol of substrate turned over/minute. All enzymatic reactions were performed in 50 mM triethanolamine (TEA) buffer (pH 7.4) with 2.5 mM NAD⁺. To prepare the probe solution, AlDeLuc was dissolved in DMSO and incubated with 1 M HCl. After an hour, this solution was neutralized with 1 M NaOH, and diluted with PBS. To a clear 24-well plate, TEA buffer, NAD⁺, the ALDH isoform, and then AlDeLuc (2 μM) were added sequentially to a final volume of 1 mL. After an hour incubation at room temperature, ATP-MgSO₄ (45 μL, prepared by mixing 17.78 mM ATP-Mg and 35.56 mM MgSO₄ in a 1:1 ratio in PBS buffer) and luciferase (5 μL, 1 mg/mL in PBS containing 10% glycerol) were added to initiate bioluminescence production. The wells were immediately imaged using the IVIS imaging system in open mode. ROIs were drawn around each well and the signal intensity was quantified using the Living Image Analysis. The signal enhancement was normalized relative to the ALDH2 isoform.

Dose-dependent Enhancement of AlDeLuc. The response of ALDH1A1 (0.4 μM) against different concentrations of AlDeLuc was measured in a black 96-well plate and was monitored using the IVIS imaging system. Various stocks of AlDeLuc were generated so that 20 μL of the probe solution gave the desired concentration. Stocks were prepared by the following: AlDeLuc was first dissolved in DMSO followed by a 60 minute incubation in equal volume 1 M HCl. Subsequently, the reaction was neutralized with 1 M NaOH and diluted with PBS. To a black 96-well plate, TEA buffer (pH 7.4), NAD⁺ (2.5 mM), and either 2 M, 5 μM, 10 μM, or 50 μM of AlDeLuc was added. After 60 minutes, each well was treated with ATP-MgSO₄ (45 μL, prepared by mixing 17.78 mM ATP-Mg and 35.56 mM MgSO₄ in a 1:1 ratio in PBS buffer) and luciferase (5 μL, 1 mg/mL in PBS containing 10% glycerol) to initiate bioluminescence production. Light was collected immediately after mixing, and the signal enhancement was determined relative to control wells not treated with enzymes. The total volume of each well was 200 μL (TEA (pH 7.4) with 0.2% DMSO). All data reported as the mean±standard deviation (n=4).

Dose-dependent Activation of AlDeLuc by ALDH1A1. The response of AlDeLuc (5 μM) against different concentrations of ALDH1A1 was measured in a black 96-well plate and was monitored using the IVIS imaging system. To a black 96-well plate, PBS (pH 7.4), NAD⁺, and either 0.05 μM, 0.1 μM, 0.4 μM, 2 μM, or 4 μM of ALDH1A1, was added AlDeLuc. Immediately after, each well was treated with ATP-MgSO₄ (45 μL, prepared by mixing 17.78 mM ATP-Mg and 35.56 mM MgSO₄ in a 1:1 ratio in PBS buffer) and luciferase (5 μL, 1 mg/mL in PBS containing 10% glycerol) to initiate bioluminescence production. Light was collected immediately after mixing, and the signal enhancement was determined relative to control wells not treated with enzymes. The total volume of each well was 200 μL (PBS (pH 7.4) with 0.2% DMSO). All data reported as the mean standard deviation (n=3).

Stability of AlDeLuc at Acidic, Neutral and Alkaline pHs. The stability of AlDeLuc (2 μM) to acidic, neutral and alkaline conditions were investigated in a black 96-well plate and monitored using the IVIS imaging system. AlDeLuc was incubated in 1 M HCl (acidic), 1 M NaCl (neutral, pH=7), or 1 M NaOH (alkaline) for one hour, neutralized, and diluted with 0.1 M potassium phosphate buffer (pH 7.4). To the black well-plate was added PBS (pH 7.4), NAD⁺ (1 mM), ALDH1A1 (0.4 μM), and either the acidic-, neutral-, or alkaline-treated AlDeLuc. Immediately after, each well was treated with ATP-MgSO₄ (45 μL, prepared by mixing 17.78 mM ATP-Mg and 35.56 mM MgSO₄ in a 1:1 ratio in PBS buffer) and luciferase (5 μL, 1 mg/mL in PBS containing 10% glycerol) to initiate bioluminescence production. Light was collected immediately after mixing, and the signal enhancement was determined relative to control wells not treated with AlDeLuc. The total volume of each well was 200 μL (PBS (pH 7.4) with 0.2% DMSO). All data reported as the mean±standard deviation.

Exploring the Stability of AlDeLuc to β-Galactosidase. The stability of AlDeLuc (10 μM) against β-Galactosidase (β-gal) was determined using a black 96-well plate and monitored using the IVIS imaging system. In a 96-well plate was added PBS (pH 7.4), β-gal (1 unit), and AlDeLuc. As a positive control to the same plate, was added PBS (pH 7.4), ALDH1A1 (0.4 μM), NAD⁺ (1 mM), and acid activated AlDeLuc. Immediately after AlDeLuc was added, ATP-MgSO₄ (45 μL, prepared by mixing 17.78 mM ATP-Mg and 35.56 mM MgSO₄ in a 1:1 ratio in PBS buffer) and luciferase (5 μL, 1 mg/mL in PBS containing 10% glycerol) were administered to initiate bioluminescence production. Light was collected promptly after mixing, and the total luminescent counts were measured for both conditions. The total volume of each well was 200 μL with 0.1% DMSO. All data reported as the mean±standard deviation (n=4).

Investigating the Stability of AlDeLuc to Rat Liver Microsomes. The stability of AlDeLuc (2 μM) in rat liver microsomes (RLM) was measured in a black 96-well plate and was monitored using the IVIS imaging system. To Eppendorf tubes containing potassium phosphate buffer (0.1 M, pH 7.4), RLM (0.25 ng/mL or 5 ng/mL), and AlDeLuc (2 μM) was added NADPH (50 μM in potassium phosphate buffer) and incubated at 37° C. for 15 minutes. In parallel, Eppendorf tubes with potassium phosphate buffer (0.1 M, pH 7.4), ALDH1A1 (0.4 μM), NAD⁺ (1 mM), and AlDeLuc (2 μM, activated with acid) were warmed at 37° C. for 15 minutes. The solutions were transported to the laboratory housing the IVIS imaging system (˜10 minutes), transferred to a 96-well plate, and immediately administered ATP-MgSO₄ (45 μL, prepared by mixing 17.78 mM ATP-Mg and 35.56 mM MgSO₄ in a 1:1 ratio in PBS buffer) and luciferase (5 μL, 1 mg/mL in PBS containing 10% glycerol) to initiate bioluminescence production. Light was collected immediately after mixing, and the total luminescent counts were collected in each condition. The total volume of each well was 200 μL with 0.2% DMSO. All data reported as the mean±standard deviation (n=4).

Validation of ALDH-Mediated Activation of AlDeLuc in Human Lung Cancer Cells. A549 cells were cultured in T75 culture flasks. After two days, cells had become ˜90% confluent. An aliquot of HAMS FK12 media (1 mL) was removed from each flask and replaced with either disulfiram (DSF) (100 μM, 0.5% DMSO) or vehicle (0.5% DMSO). After incubation for one hour, the solutions were aspirated, the cells were washed with PBS, and resuspended in fresh HAMS FK12 media (9 mL). Each flask was then incubated with the AlDeLuc such that the final concentration of the probe is 100 M and 1% DMSO. After 60 minutes this solution was aspirated, washed with fresh PBS, and detached from the culture flasks. The cells were then transferred to a 15 mL centrifuge tube and pelleted at 1000 rpm for 5 minutes at room temp. The cells were resuspended in PBS along with 10% protease inhibitor solution (1 protease inhibitor mini tablet per 10 mL of PBS, Pierce, Thermo Fisher Scientific) and sonicated on ice for 2.5 minutes (pulse 01, 01, 40%). The cell debris was removed via centrifugation, the supernatant was plated (95 μL/well) into a 96-well plate (n=4 for each condition), and treated with luciferase (5 μL, 1 mg/mL) and immediately imaged using the IVIS imaging system in open mode. ROIs were drawn around each well and the signal intensity was quantified using the Living Image Analysis software.

Validation of ALDH-Mediated Activation of AlDeLuc in Murine Breast Cancer Cells. 4T1 cells were cultured in T75 culture flasks. After two days, cells had become ˜90% confluent. An aliquot of RPMI 1640 media (1 mL) was removed from each flask and replaced with either DEAB (500 M, 0.5% DMSO) or vehicle (0.5% DMSO). After incubation for one hour, the solutions were aspirated, washed with PBS, and resuspended in fresh RPMI 1640 media (9 mL). Each flask was then incubated with the AlDeLuc (100 μM) and DEAB (500 μM) or AlDeLuc (100 μM) and vehicle (DMSO), each with a final concentration of 1% DMSO. After 60 minutes, this solution was aspirated, washed with fresh PBS, and detached from the culture flasks. The cells were then transferred to a 15 mL centrifuge tube and pelleted at 1000 rpm for 5 minutes at room temp. The cells were resuspended in PBS along with 10% protease inhibitor solution (1 protease inhibitor mini tablet per 10 mL of PBS, Pierce, Thermo Fisher Scientific) and sonicated on ice for 2.5 minutes (pulse 01, 01, 40%). The cell debris was removed via centrifugation and the supernatant was plated (95 L/well) into a 96-well plate (n=4 for each condition). Each well was treated with luciferase (5 μL, 1 mg/mL) and immediately imaged using the IVIS imaging system in open mode. ROIs were drawn around each well and the signal intensity was quantified using the Living Image Analysis software.

Influence of L-NMMA on ALDH1A1 activity in 4T1 Cells Using AlDeLuc. 4T1 cells were cultured in T75 culture flasks. After two days, cells had become ˜90% confluent. The media was aspirated and 9 mL of fresh media was added. The flasks were then treated with L-NMMA (1 mM, cell culture water) or vehicle (cell culture water). After incubation for 60 minutes, the solutions were aspirated, washed with PBS, and resuspended in fresh RPMI 1640 media (9 mL). AlDeLuc was added to each flask (50 M, 5% v/v DMSO:PBS) for a final concentration of 0.5% DMSO per flask. After 60 minutes this solution was aspirated, washed with fresh PBS, and detached from the culture flasks. The cells were then transferred to a 15 mL centrifuge tube and pelleted at 1000 rpm for 5 minutes at room temp. The cells were resuspended in PBS along with 10% protease inhibitor solution (1 protease inhibitor mini tablet per 10 mL of PBS, Pierce, Thermo Fisher Scientific) and sonicated on ice for 2.5 minutes (pulse 01, 01, 40%). The cell debris was removed via centrifugation and the supernatant was plated (95 L/well) into a 96-well plate (n=4 for each condition). Each well was treated with luciferase (5 μL, 1 mg/mL) and immediately imaged using the IVIS imaging system in open mode. ROIs were drawn around each well and the signal intensity was quantified using the Living Image Analysis software.

Exploring the Effect of Nitric Oxide on ALDH1A1 activity in 4T1 Cells Using AlDeLuc. 4T1 cells were cultured in T75 flasks, and once they reached ˜30% confluency (1 day), the media was removed and replaced with fresh RPMI 1640 media supplemented with either DETA NONOate (50 μM) or vehicle (media). After 24 hours, the media was aspirated, and the cells were treated once more with RPMI 1640 media containing either DETA NONOate (50 μM) or vehicle (media). After 27 hours, the suspension was removed and cells were treated with AlDeLuc (2 μM, final concentration of 0.01% DMSO). After 60 minutes the AlDeLuc solution was aspirated, washed with fresh PBS, and detached from the culture flasks. The cells were then transferred to a 15 mL centrifuge tube and pelleted at 1000 rpm for 5 minutes at room temp. The cells were resuspended in PBS along with 10% protease inhibitor solution (1 protease inhibitor mini tablet per 10 mL of PBS, Pierce, Thermo Fisher Scientific) and sonicated on ice for 2.5 minutes (pulse 01, 01, 40%). The cell debris was removed via centrifugation and the supernatant was plated (95 μL/well) into a 96-well plate (n=4 for each condition). Each well was treated with luciferase (5 μL, 1 mg/mL) and immediately imaged using the IVIS imaging system in open mode. ROIs were drawn around each well and the signal intensity was quantified using the Living Image Analysis software.

Determining Sensitivity of AlDeLuc by Cell Concentration Changes. A549 cells were cultured in T75 flasks, after they had become ˜80% confluent (3 days), they were detached, pelleted (1000 rpm, room temp, 5 minutes) and diluted to a concentration of 500,000 cells/mL. This solution of cells 10 underwent five-fold serial dilutions with HAMS-FK12 media (producing stocks of 250,000 cells/mL, 125,000 cells/mL, 62,500 cells/mL, 31,250 cells/mL, and 15,625 cells/mL). In a 6-well plate, 2 mL of each stock was used to seed three wells (3 plates total). After 16 hours, the media was removed and replaced with a solution of AlDeLuc in serum-free media (125 μM, 5% DMSO), and incubated at 37° C. for 60 minutes. The cells were then washed with PBS (500 μL), detached, and pelleted (1000 rpm, room temp, 5 minutes). They were resuspended in PBS along with 10% protease inhibitor solution (1 protease inhibitor mini tablet per 10 mL of PBS, Pierce, Thermo Fisher Scientific) and sonicated on ice for 2.5 minutes (pulse 01, 01, 40%). The cell debris was removed via centrifugation and the supernatant was plated (95 μL/well) into a 96-well plate (n=3 for each concentration of cells). Each well was treated with luciferase (5 μL, 1 mg/mL) and immediately imaged using the IVIS imaging system in open mode. ROIs were drawn around each well and the signal intensity was quantified using the Living Image Analysis software.

Influence of Interferon-gamma (INF-γ) on ALDH1A1 activity in 4T1 Cells Using AlDeLuc. 4T1 cells were cultured in T75 culture flasks. After two days, cells had become ˜90% confluent. An aliquot of RPMI 1640 was removed (26.8 μL) and the flasks were then treated with INF-γ (26.8 μL of 10 g/mL stock in PBS, 26.8 ng/mL final conc.) or vehicle (26.8 μL PBS). After 12 hours, the solutions were aspirated, washed with PBS, and resuspended in fresh RPMI 1640 media (9 mL). AlDeLuc was added to each flask (50 M, 5% v/v DMSO:PBS) for a final concentration of 0.5% DMSO per flask. After 60 minutes this solution was aspirated, washed with fresh PBS, and detached from the culture flasks. The cells were then transferred to a 15 mL centrifuge tube and pelleted at 1000 rpm for 5 minutes at room temp. The cells were resuspended in PBS along with 10% protease inhibitor solution (1 protease inhibitor mini tablet per 10 mL of PBS, Pierce, Thermo Fisher Scientific) and sonicated on ice for 2.5 minutes (pulse 01, 01, 40%). The cell debris was removed via centrifugation and the supernatant was plated (95 μL/well) into a 96-well plate (n=4 for each condition). Each well was treated with luciferase (5 μL, 1 mg/mL) and immediately imaged using the IVIS imaging system in open mode. ROIs were drawn around each well and the signal intensity was quantified using the Living Image Analysis software.

Generation of 4T1-Luc Breast Cancer Model. Female or male BALB/c mice (˜6 weeks old) were inoculated with 4T1-Luc cells (50 μL of 1×10⁶ cells in 1:1 v/v serum-free RPMI 1640 media and Matrigel) via subcutaneous injections into each flank. Tumor volumes were measured using the caliper method every three days. Body weight was measured daily over the course of the experiment. After two weeks, the tumor was in the 200 to 400 mm³ range.

Generation of an Orthotopic 4T1-Luc Breast Cancer Model for Diet Studies. Female BALB/c mice (˜6 weeks old) were inoculated with 4T1-Luc cells (50 μL of 1×10⁵ cells in 1:1 v/v serum-free RPMI 1640 media and Matrigel) via subcutaneous injections into the mammary fat pad. Tumor volumes were measured using the caliper method every three days. Body weight was measured daily over the course of the experiment.

Bioluminescence Imaging of a Heterotopic Breast Cancer Model with AlDeLuc. BALB/c mice bearing 4T1-Luc tumors were anesthetized using isoflurane (1-3% for maintenance; up to 5% for induction) in oxygen from a precision vaporizer. After testing to ensure animals are fully under anesthesia, each mouse was administered a control solution of 1:19 v/v DMSO:PBS via retro-orbital injection. After 40 minutes, the mice were imaged on the IVIS imaging system. Luminescence was collected using an open filter set. ROIs were drawn around both the left and right tumors, and the intensity was calculated using the Living Image Analysis software.

The following day the mice were anesthetized, and administered AlDeLuc (0.55 mg/kg, 1:19 v/v DMSO:PBS). After 40 minutes, the mice were imaged on the IVIS imaging system. Luminescence was collected using an open filter set. ROIs were drawn around both the left and right tumors, and the intensity was calculated using the Living Image Analysis software.

Validation of ALDH-mediated Activation of AlDeLuc in a Heterotopic Breast Cancer Model via DEAB Inhibition. BALB/c mice bearing 4T1-Luc tumors were anesthetized using isoflurane (1-3% for maintenance; up to 5% for induction) in oxygen from a precision vaporizer. After testing to ensure animals are fully under anesthesia, each mouse was administered AlDeLuc via retro-orbital injection (0.55 mg/kg, 1:19 v/v DMSO:PBS). Images were captured using the IVIS imaging system 40 minutes after injection. Bioluminescence light was captured in open mode (no filters were applied). ROIs were drawn around each tumor and summed for each animal. The signal intensity was quantified using the Living Image Analysis Software. After two days, the same animals were anesthetized and co-administered a solution of AlDeLuc and DEAB (0.55 mg/kg and 93 mg/kg respectively, 1:19 v/v DMSO:PBS). After the allotted 40 minutes, the images were captured using the IVIS imaging system. Bioluminescence was collected in open mode (no filters were applied). ROIS were drawn around each tumor and summed for each animal. The signal intensity was quantified using the Living Image Analysis Software.

Evaluation of Systemic Toxicity of AlDeLuc in BALB c mice. BALB/c mice were anesthetized and administered AlDeLuc or vehicle via retro-orbital injection (0.55 mg/kg, 1:19 v/v DMSO:PBS). After four hours, mice were euthanized, and their organs were harvested, fixed in formalin, and submitted to Veterinary Medicine for Hematoxylin and eosin staining.

### Example 2. Pharmaceutical Dosage Forms

The following formulations illustrate representative pharmaceutical dosage forms that may be used for the therapeutic or prophylactic administration of a compound of a formula described herein, a compound specifically disclosed herein, or a pharmaceutically acceptable salt or solvate thereof (hereinafter referred to as ‘Compound X’):

| (i) Tablet 1 | mg/tablet |
|---|---|
| ‘Compound X’ | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| ‘Compound X’ | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| ‘Compound X’ | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/mL) | mg/mL |
|---|---|
| ‘Compound X’ (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |

| (v) Injection 2 (10 mg/mL) | mg/mL |
|---|---|
| ‘Compound X’ (free acid form) | 10.0 |
| Monobasic sodium phosphate | 0.3 |
| Dibasic sodium phosphate | 1.1 |
| Polyethylene glycol 400 | 200.0 |
| 0.1N Sodium hydroxide solution | q.s. |
| (pH adjustment to 7.0-7.5) | |
| Water for injection | q.s. ad 1 mL |

| (vi) Aerosol | mg/can |
|---|---|
| ‘Compound X’ | 20 |
| Oleic acid | 10 |
| Trichloromonofluoromethane | 5,000 |
| Dichlorodifluoromethane | 10,000 |
| Dichlorotetrafluoroethane | 5,000 |

| (vii) Topical Gel 1 | wt. % |
|---|---|
| ‘Compound X’ | 5% |
| Carbomer 934 | 1.25% |
| Triethanolamine | q.s. |
| (pH adjustment to 5-7) | |
| Methyl paraben | 0.2% |
| Purified water | q.s. to 100 g |

| (viii) Topical Gel 2 | wt. % |
|---|---|
| ‘Compound X’ | 5% |
| Methylcellulose | 2% |
| Methyl paraben | 0.2% |
| Propyl paraben | 0.02% |
| Purified water | q.s. to 100 g |

| (ix) Topical Ointment | wt. % |
|---|---|
| ‘Compound X’ | 5% |
| Propylene glycol | 1% |
| Anhydrous ointment base | 40% |
| Polysorbate 80 | 2% |
| Methyl paraben | 0.2% |
| Purified water | q.s. to 100 g |

| (x) Topical Cream 1 | wt. % |
|---|---|
| ‘Compound X’ | 5% |
| White bees wax | 10% |
| Liquid paraffin | 30% |
| Benzyl alcohol | 5% |
| Purified water | q.s. to 100 g |

| (xi) Topical Cream 2 | wt. % |
|---|---|
| ‘Compound X’ | 5% |
| Stearic acid | 10% |
| Glyceryl monostearate | 3% |
| Polyoxyethylene stearyl ether | 3% |
| Sorbitol | 5% |
| Isopropyl palmitate | 2% |
| Methyl Paraben | 0.2% |
| Purified water | q.s. to 100 g |

These formulations may be prepared by conventional procedures well known in the pharmaceutical art. It will be appreciated that the above pharmaceutical compositions may be varied according to well-known pharmaceutical techniques to accommodate differing amounts and types of active ingredient ‘Compound X’. Aerosol formulation (vi) may be used in conjunction with a standard, metered dose aerosol dispenser. Additionally, the specific ingredients and proportions are for illustrative purposes. Ingredients may be exchanged for suitable equivalents and proportions may be varied, according to the desired properties of the dosage form of interest.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

What is claimed is:

1. A compound of formula I: 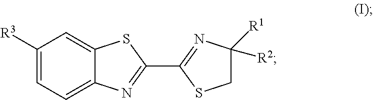
or a salt thereof,
wherein
R¹ is —CH(OR^{a})₂, —CHO, or —CO₂R^{b};
R² is H; or
R¹ and R² taken together with the carbon atom to which they are attached is: 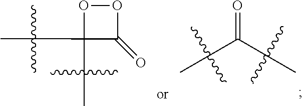
R³ is —N(R^{c})² or —SR^{c};
R^{a} is each independently —(C₁-C₆)alkyl or H;
R^{b} is H or adenosine monophosphate (AMP); and
R^{c} is each independently —(C₁-C₆)alkyl;
wherein each —(C₁-C₆)alkyl can independently be unbranched —(C₃-C₆)alkyl or branched —(C₃-C₆)alkyl.

2. The compound of claim 1, wherein R¹ is —CH(OR^{a})₂.

3. The compound of claim 1, wherein each R^{a} is —CH₃, —CH₂CH₃, or —CH(CH₃)₂.

4. The compound of claim 1, wherein R³ is —N(R^{c})².

5. The compound of claim 1, wherein each R^{c} is —CH₂CH₃.

6. The compound of claim 1, wherein the carbon atom to which R¹ and R² are attached has an (S)-configuration.

7. The compound of claim 1, wherein the carbon atom to which R¹ and R² are attached has an (R)-configuration.

8. The compound of claim 1, represented by formula II: 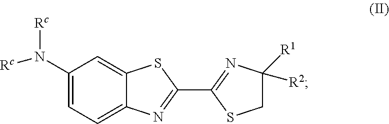
or a salt thereof.

9. The compound of claim 1, represented by formula III: 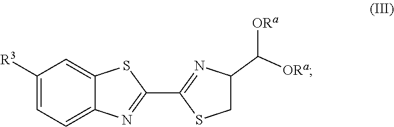
or a salt thereof.

10. The compound of claim 1, represented by formula IV: 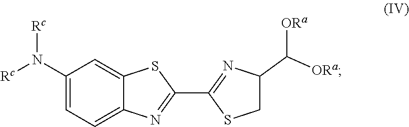
or a salt thereof.

11. The compound of claim 1, wherein the compound is AlDeLuc: 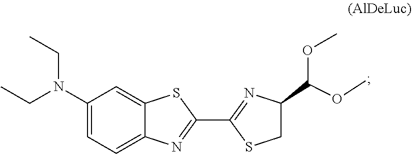 [SMILES: CCN(CC)c1ccc2nc(C3=N[C@@H](C(OC)OC)CS3)sc2c1]
or a salt thereof.

12. The compound of claim 1, wherein the compound is: 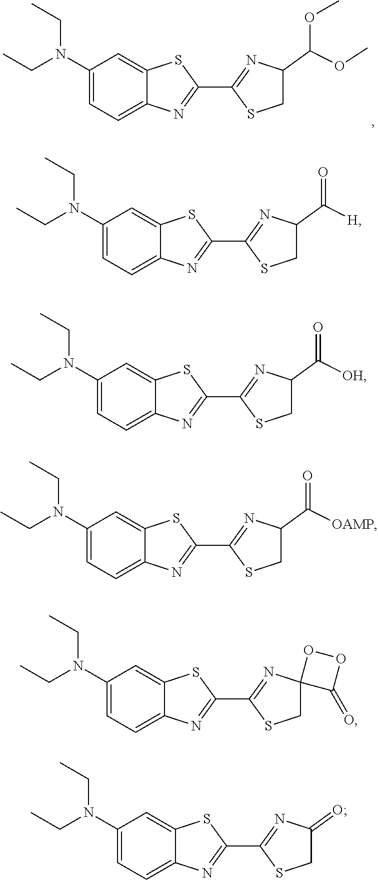
or a salt thereof.

13. A composition comprising a compound of claim 1 and pharmaceutically acceptable diluent or carrier.

14. A method for detecting cancer stem cells (CSCs), comprising:
a) contacting CSCs comprising an aldehyde dehydrogenase (ALDH), an acid, and a compound of formula I: 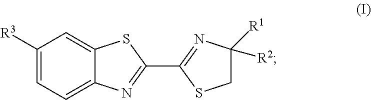
or a salt thereof,
wherein
R¹ is —CH(OR^{a})₂, —CHO, or —CO₂R^{b};
R² is H; or
R¹ and R² taken together with the carbon atom to which they are attached is: 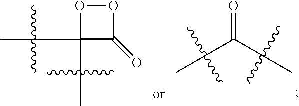
R³ is —N(R^{c})², —OR^{c}, or —SR;
R^{a} is each independently —(C₁-C₆)alkyl or H;
R^{b} is H or adenosine monophosphate (AMP); and
R^{c} is each independently —(C₁-C₆)alkyl;
wherein each —(C₁-C₆)alkyl can independently be unbranched —(C₃-C₆)alkyl or branched —(C₃-C₆)alkyl; and
b) detecting a bioluminescent signal emitted from the CSCs;
wherein, a compound of formula I comprising the acetal moiety R¹ is —CH(OR^{a})₂ is hydrolyzed by the acid to an aldehyde, and the aldehyde is oxidized by the ALDH to liberate a carboxylic acid substrate for luciferase;
wherein the bioluminescent signal emitted from the CSCs is thereby detected.

15. The method of claim 14, wherein the compound is (S)-2-(4-(dimethoxymethyl)-4,5-dihydrothiazol-2-yl)-N,N-diethylbenzo[d]thiazol-6-amine (AlDeLuc).

16. The method of claim 14, wherein the aldehyde dehydrogenase is the ALDH₁A₁ isoform.

17. The method of claim 14, wherein the luciferase catalyzes a conversion of the carboxylic acid substrate to 2-(6-(diethylamino)benzo[d]thiazol-2-yl)thiazol-4(5H)-one that yields a bioluminescent signal.

18. The method of claim 14, wherein a tumor comprises the cancer stem cells.

19. The method of claim 18, wherein the tumor comprises the acid in an endosomal compartment within the cancer stem cells.

20. The method of claim 14, wherein the aldehyde is 2-(6-(diethylamino)benzo[d]thiazol-2-yl)-4,5-dihydrothiazole-4-carbaldehyde and/or the carboxylic acid substrate is 2-(6-(diethylamino)benzo[d]thiazol-2-yl)-4,5-dihydrothiazole-4-carboxylic acid.
